(19) United States
(12) Patent Application Publication
(10) Pub. No.: US 2024/0001344 A1
(43) Pub. Date: Jan. 4, 2024

(54) Processes for Calcining a Catalyst

(71) Applicant: ExxonMobil Chemical Patents Inc., Baytown, TX (US)

(72) Inventors: Xiaoying Bao, Houston, TX (US); Chuansheng Bai, Phillipsburg, NJ (US)

(21) Appl. No.: 18/208,929

(22) Filed: Jun. 13, 2023

Related U.S. Application Data
(60) Provisional application No. 63/357,729, filed on Jul. 1, 2022.

(51) Int. Cl. B01J 23/62; B01J 23/02; B01J 37/08; B01J 37/18; B01J 37/14

(52) U.S. Cl. CPC B01J 23/626; B01J 23/02; B01J 37/08; B01J 37/18; B01J 37/14

(57) ABSTRACT

Processes for calcining a catalyst. The process can include subjecting a synthesized catalyst that includes Pt disposed on a support to an initial calcination that includes exposing the catalyst to a first reducing gas or a first oxidizing gas to produce an initial calcined catalyst. The process can optionally include subjecting the initial calcined. catalyst to a cycle calcination that includes exposing the initial calcined catalyst to a second reducing gas and a second oxidizing gas to produce a cycle calcined catalyst. The process can optionally include subjecting the initial or the cycle calcined catalyst to a final calcination that includes exposing the initial or the cycle calcined catalyst to a third reducing gas or a third oxidizing gas. At least one of the cycle and the final calcination can be carried out. A calcined catalyst can be obtained at the end of the cycle or the final calcination.

### CROSS-REFERENCE TO RELATED APPLICATION

This application claims priority to and the benefit of U.S. Provisional Application No. 63/357,729 having a filing date of Jul. 1, 2022, the disclosure of which is incorporated herein by reference in its entirety.

### FIELD

This disclosure relates to processes for calcining a synthesized catalyst. More particularly, this disclosure relates to calcining a synthesized catalyst that includes Pt disposed on a support to produce a calcined catalyst.

### BACKGROUND

Catalytic reforming or dehydrogenation, dehydroaromatization, and/or dehydrocyclization of alkane and/or alkyl aromatic hydrocarbons are industrially important chemical conversion processes that are endothermic and equilibrium-limited. The reforming or dehydrogenation dehydroaromatization, and/or dehydrocyclization of alkanes, C₁-C₁₂ alkanes, and/or alkyl aromatics, e.g., ethylbenzene, can be done through a variety of different catalysts such as the Pt-based, Ni-based, Pd-based, Ru-based, Re-based, Cr-based, Ga-based, V-based, Zr-based, In-based, W-based, Mo-based, Zn-based, and Fe-based systems.

A catalyst, after synthesis, typically needs to be pre-treated or conditioned before the synthesized catalyst can be used in a commercial reactor. One conditioning process includes equilibration, usually at room temperature with flowing or stagnant gas, to allow any liquid precursors to diffuse into the catalyst. Another conditioning process includes drying, usually at a temperature less than calcination with a flowing gas or in vacuum, to allow most volatile components to leave the catalyst. Another conditioning process includes calcination, usually done at a temperature higher than drying with a flowing gas or in vacuum, to allow pre-cursors in the catalyst to transform into active species or species that are structurally/chemically closer to the active species. While these conditioning processes improve the performance of an as synthesized catalyst, such improvement is less than desirable.

There is a need, therefore, for improved processes for conditioning a synthesized catalyst. This disclosure satisfies this and other needs.

### SUMMARY

Processes for calcining a synthesized catalyst are provided. In some embodiments, the process for calcining a catalyst can include subjecting a synthesized catalyst that includes Pt disposed on a support to an initial calcination that includes exposing the synthesized catalyst to a first reducing gas under reduction conditions or a first oxidizing gas under oxidation conditions to produce an initial calcined catalyst. The synthesized catalyst can include <0.05 wt % of the Pt, based on the non-volatile weight of the catalyst. The process can optionally include subjecting the initial calcined catalyst to a cycle calcination that can include exposing the initial calcined catalyst to a second reducing gas under reduction conditions and a second oxidizing gas under oxidation conditions for n cycles to produce a cycle calcined catalyst. The variable n can be a whole number. The cycle calcination can start with the second oxidizing gas when the initial calcination uses the first reducing gas. The cycle calcination can start with the second reducing gas when the initial calcination uses the first oxidizing gas. When n is ≥2, a composition of the second reducing gas used in each cycle calcination can be the same or different and a composition of the second oxidizing gas used in each cycle calcination can be the same or different. The process can optionally include subjecting the initial calcined catalyst or the cycle calcined catalyst to a final calcination that can include exposing the initial calcined catalyst or the cycle calcined catalyst to a third reducing gas under reduction conditions or a third oxidizing gas under oxidation conditions. At least one of the cycle calcination and the final calcination can be carried out. The final calcination, when carried out, can use the third oxidizing gas when the initial calcination uses the first reducing gas or, when carried out, the cycle calcination ends with the second reducing gas. The final calcination, when carried out, can use the third reducing gas when the initial calcination uses the first oxidizing gas or, when carried out, the cycle calcination ends with the second oxidizing gas. The reduction conditions used in the initial calcination, the optional cycle calcination, and the optional final calcination independently can include heating the catalyst at a temperature in a range from 500° C. to 850° C. for a time period in a range from 30 seconds to 10 hours. The oxidizing conditions used in the initial calcination, the optional cycle calcination, and the optional final calcination independently can include heating the catalyst at a temperature in a range from 350° C. to 850° C. for a time period in a range from 30 seconds to 10 hours. A calcined catalyst can be obtained at the end of the cycle calcination or at the end of the final calcination.

In some embodiments, the process for calcining a catalyst can include subjecting synthesized catalyst particles that can include Pt disposed on a support to an initial calcination that can include exposing the catalyst particles to a first reducing gas under reduction conditions or a first oxidizing gas under oxidation conditions to produce initial calcined catalyst particles. The synthesized catalyst particles can have a size and particle density that is consistent with a Geldart A definition of a fluidizable solid. The process can optionally include subjecting the initial calcined catalyst particles to a cycle calcination that can include exposing the initial calcined catalyst particles to a second reducing gas under reduction conditions and a second oxidizing gas under oxidation conditions for n cycles to produce cycle calcined catalyst particles. The variable n can be a whole number. The cycle calcination can start with the second oxidizing gas when the initial calcination uses the first reducing gas. The cycle calcination can start with the second reducing gas when the initial calcination uses the first oxidizing gas. When n is ≥2, a composition of the second reducing gas used in each cycle calcination can be the same or different and a composition of the second oxidizing gas used in each cycle calcination can be the same or different. The process can optionally include subjecting the initial calcined catalyst particles or the cycle calcined catalyst particles to a final calcination that can include exposing the initial calcined catalyst particles or the cycle calcined catalyst particles to a third reducing gas under reduction conditions or a third oxidizing gas under oxidation conditions. At least one of the cycle calcination and the final calcination can be carried out. The final calcination, when carried out, can use the third oxidizing gas when the initial calcination uses the first reducing gas or, when carried out, the cycle calcination ends with the second reducing gas. The final calcination, when carried out, can use the third reducing gas when the initial calcination uses the first oxidizing gas or, when carried out, the cycle calcination ends with the second oxidizing gas. The reduction conditions used in the initial calcination, the optional cycle calcination, and the optional final calcination independently can include heating the catalyst particles at a temperature in a range from 500° C. to 850° C. for a time period in a range from 30 seconds to 10 hours. The oxidizing conditions used in the initial calcination, the optional cycle calcination, and the optional final calcination independently can include heating the catalyst particles at a temperature in a range from 350° C. to 850° C. for a time period in a range from 30 seconds to 10 hours. Calcined catalyst particles can be obtained at the end of the cycle calcination or at the end of the final calcination.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step,” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step, Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “a reactor” or “a conversion zone” include embodiments where one, two or more reactors or conversion zones are used, unless specified to the contrary or the context clearly indicates that only one reactor or conversion zone is used.

The term “hydrocarbon” means (i) any compound consisting of hydrogen and carbon atoms or (ii) any mixture of two or more such compounds in (i), The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm-Cn hydrocarbon,” where m and n are positive integers and m<n, means any of Cm, Cm+1, Cm+2, . . . , Cn-1, Cn hydrocarbons, or any mixtures of two or more thereof. Thus, a “C2 to C3 hydrocarbon” or “C2-C3 hydrocarbon” can be any of ethane, ethylene, acetylene, propane, propene, propene, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+hydrocarbon” means (i) any hydrocarbon compound comprising carbon atom(s) in its molecule at the total number of at least n, or (ii) any mixture of two or more such hydrocarbon compounds in (i). A “Cn-hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

For the purposes of this disclosure, the nomenclature of elements is pursuant to the version of the Periodic Table of Elements (under the new notation) as provided in Hawley's Condensed Chemical Dictionary, 16^{th} Ed., John Wiley & Sons, Inc., (2016), Appendix V. For example, a Group 2 element includes Mg, a Group 8 element includes Fe, a Group 9 element includes Co, a Group 10 element includes Ni, and a Group 13 element includes Al. The term “metalloid”, as used herein, refers to the following elements: B, Si, Ge, As, Sb, Te, and At. In this disclosure, when a given element is indicated as present, it can be present in the elemental state or as any chemical compound thereof, unless it is specified otherwise or clearly indicated otherwise by the context.

The term “alkane” means a saturated hydrocarbon. The term “cyclic alkane” means a saturated hydrocarbon comprising a cyclic carbon ring in the molecular structure thereof. An alkane can be linear, branched, or cyclic.

The term “aromatic” is to be understood in accordance with its art-recognized scope, which includes alkyl substituted and unsubstituted mono- and polynuclear compounds.

The term “rich” when used in phrases such as “X-rich” or “rich in X” means, with respect to an outgoing stream obtained from a device, e.g., a conversion zone, that the stream comprises material X at a concentration higher than in the feed material fed to the same device from which the stream is derived. The term “lean” when used in phrases such as “X-lean” or “lean in X” means, with respect to an outgoing stream obtained from a device, e.g., a conversion zone, that the stream comprises material X at a concentration lower than in the feed material fed to the same device from which the stream is derived.

The term “mixed metal oxide” refers to a composition that includes oxygen atoms and at least two different metal atoms that are mixed on an atomic scale. For example, a “mixed Mg/Al metal oxide” has O, Mg, and Al atoms mixed on an atomic scale and is substantially the same as or identical to a composition obtained by calcining an Mg/Al hydrotalcite that has the general chemical formula

$$[ {Mg}_{( 1 - x )} {{Al}_{x} ( OH )}_{2} ] ( {A}_{\frac{x}{n}}^{n -} ) \cdot {m H}_{2} O ] ,$$

where A is a counter anion of a negative charge n, x is in a range of from >0 to <1, and m is ≥0. A material consisting of nm sized MgO particles and nm sized Al₂O₃ particles mixed together is not a mixed metal oxide because the Mg and Al atoms are not mixed on an atomic scale but are instead mixed on a nm scale.

The terms “calcination” and “calcining” refer to heating a material, e.g., synthesized catalyst or a support, to a temperature of 350° C. or more under any atmosphere, e.g., an oxidizing atmosphere, an inert atmosphere, or a reducing atmosphere. The term “calcined” refers to a material, e.g., a synthesized catalyst or a support, that has been subjected to calcination calcining.

The term “selectivity” refers to the production (on a carbon mole basis) of a specified compound in a catalytic reaction. As an example, the phrase “an alkarie hydrocarbon conversion reaction has a 100% selectivity for an olefin hydrocarbon” means that 100% of the alkane hydrocarbon (carbon mole basis) that is converted in the reaction is converted to the olefin hydrocarbon. When used in connection with a specified reactant, the term “conversion” means the amount of the reactant consumed in the reaction. For example, when the specified reactant is propane, 100% conversion means 100% of the propane is consumed in the reaction. In another example, when the specified reactant is propane, if one mole of propane converts to one mole of methane and one mole of ethylene, the selectivity to methane is 33.3% and the selectivity to ethylene is 66.7%. Yield (carbon mole basis) is conversion times selectivity.

As used herein, “sccm” means standard cubic centimeters per minute, which is a flow measurement used to indicate the cubic centimeters (cm³) of a gas at standard temperature and pressure passing a given point within one minute. Standard temperature and pressure (STP) refers to a temperature of 273.15 K (0° C.) and an absolute pressure of 10⁵ Pa (100 kPa. 1 bar).

In this disclosure, “A, B, . . . or a combination thereof” means “A, B, . . . or any combination of any two or more of A, B, . . . ” “A, B, . . . , or a mixture thereof” means “A, B, . . . , or any mixture of any two or more of A, B, . . . ”

### Process for Calcining a Catalyst

It has been surprisingly and unexpectedly discovered that a synthesized catalyst for use in upgrading one or more hydrocarbons, e.g., dehydrogenating alkanes to produce olefins, when first subjected to a calcination process to produce a calcined catalyst, can exhibit a significantly improved performance as compared to the synthesized catalyst not subjected to the calcination process when it is contacted with one or more alkanes under dehydrogenation conditions. In some embodiments, the synthesized catalyst can include Pt disposed on a support. In some embodiments, the synthesized catalyst can include <0.05 wt %, <0.045 wt %, <0.04 wt %, <0.035 wt %, or <0.03 wt % of the Pt, based on the non-volatile weight of the catalyst. In other embodiments, the synthesized catalyst can be in the form of catalyst particles that have a size and particle density that is consistent with a Geldart A definition of a fluidizable solid and can include 0.001 wt % to 6 wt % of the Pt, based on the non-volatile weight of the catalyst.

As used herein, the term “synthesized catalyst” refers to a catalyst that includes the Pt disposed on the support that has not been subjected to a temperature of 350° C. or more. It should be understood, however, that the support, prior to the addition of the Pt, can be subjected to temperatures of greater than 350° C., but once the Pt has been disposed on the support the synthesized catalyst is not heated to a temperature of 350° C. or more until the catalyst is subjected to the calcination process. It should also be understood that the “synthesized catalyst” can be subjected to equilibration and-'or drying so long as the “synthesized catalyst” is not heated to a temperature of 350° C. or more.

Since the synthesized catalyst has not been subjected to a temperature of 350° C. or more, the synthesized catalyst can include one or more volatile compounds adsorbed thereon and/or one or more compounds that could form volatile compound(s) and desorb at higher temperatures such as when the synthesized catalyst is heated to a temperature of 350° C. or more under an oxidizing atmosphere, a reduction atmosphere, or other atmosphere such as an inert atmosphere. As used herein, the term “non-volatile weight of the catalyst” refers to the residual weight of the synthesized catalyst or the synthesized catalyst after being conditioned in any way after being heated to a temperature of 900° C. under flowing air. The non-volatile weight of the catalyst can be quantified via thermogravimetric analysis. A typical thermogravimetric analysis procedure is as follows: 10-20 mg of the solid to be analyzed is loaded onto a platinum pan of TGA 550 from TA instruments. The weight of the solid is monitored and recorded by a micro-balance to which the platinum pan is connected. The temperature of the platinum pan and the solid can be ramped from 25° C., to 900° C. at a ramp rate of 5° C./min under a constant flow of air. The residual weight of the solid once it reaches a temperature of 900° C. is the “non-volatile weight of the solid”.

Illustrative volatile compounds can be or can include, but are not limited to, CO, H₂, CO₂, H₂O, SO₃, SO₂, HCl, H₂S, CH₄, one or more alcohols, acetone, chloroform, methylene chloride, dimethyl formamide, dimethyl sulfoxide, glycerin, ethyl acetate, or any mixture thereof. In some embodiments, if the synthesized catalyst includes CO₂ and/or H₂O, such volatile compounds can be adsorbed from the ambient environment. In some embodiments, if the synthesized catalyst includes CO₂, H₂O, one or more alcohols, acetone, chloroform, methylene chloride, dimethyl formami de, dimethyl sulfoxide, glycerin, ethyl acetate, or any mixture thereof, such volatile compounds can be adsorbed thereon during preparation of the synthesized catalyst. For example, the process for making the synthesized catalyst can include forming a slurry of the support and/or one or more compounds the support can be derived from, one or more Pt-containing compounds, and, optionally, one or more additional compounds, e.g., a promoter-containing compound, where the liquid medium includes water, one or more alcohols, and/or other liquid mediums. In some embodiments, if the metal-containing compounds added to the support contain chlorides, for example, chloroplatinic acid for platinum, tin(IV) chloride for tin, the chlorides may react with H₂O molecules to form HCl, which desorbs from the synthesized catalyst when the synthesized catalyst is heated to a temperature above 350° C. In some embodiments, if the metal-containing compounds added to the support contain sulfates, for example, tin(H) sulfate for tin, the sulfates may decompose to form SO₂, which desorbs from the synthesized catalyst when the synthesized catalyst is heated to a temperature above 350 ° C.

The process for calcining the synthesized catalyst can include subjecting the synthesized catalyst to an initial calcination that can include exposing the synthesized catalyst to a first reducing gas under reduction conditions or a first oxidizing gas under oxidation conditions to produce an initial calcined catalyst. In some embodiments, when the synthesized catalyst is subjected to the initial calcination, the synthesized catalyst can include one or more adsorbed volatile compounds. The initial calcined catalyst can have a reduced amount of adsorbed volatile compounds as compared to the synthesized catalyst.

The process for calcining the catalyst can also include at least one of two additional steps, i.e., a cycle calcination and/or a final calcination. At least one of the cycle calcination and the final calcination can be carried out. In some embodiments, the initial calcined catalyst can be subjected to the cycle calcination for n cycles, where n can be a whole number. In some embodiments, n can be equal to 1, 2, 3, 4, 5, 6, 7, 8, 9, 10, or more. In other embodiments, the initial calcined catalyst can be subjected to the final calcination. In still other embodiments, the initial calcined catalyst can be subjected to the cycle calcination followed by the final calcination. A calcined catalyst can be obtained at the end of the cycle calcination or at the end of the final calcination.

The optional cycle calcination can include exposing the initial calcined catalyst to a second reducing gas under reduction conditions and a second oxidizing gas under oxidation conditions for n cycles. The variable n is a whole number. The cycle calcination can start with the second oxidizing gas when the initial calcination uses the first reducing gas or the cycle calcination can start with the second reducing gas when the initial calcination uses the first oxidizing gas. When n is ≥2, a composition of the second reducing gas used in each cycle calcination can be the same or different and a composition of the second oxidizing. gas used in each cycle calcination can be the same or different.

In other embodiments, the process for calcining the catalyst can include subjecting the initial calcined catalyst to the optional final calcination that can include exposing the initial calcined catalyst or the cycle calcined catalyst to a third reducing gas under reduction conditions or a third oxidizing gas under oxidation conditions. The final calcination, when carried out, can use the third oxidizing gas when the initial calcination uses the first reducing gas or, when carried out, the cycle calcination ends with the second reducing gas. The final calcination, when carried out, can use the third reducing gas when the initial calcination uses the first oxidizing gas or, when carried out, the cycle calcination ends with the second oxidizing gas.

In some embodiments, the temperature in the reduction conditions used in the initial calcination, the optional cycle calcination, and the optional final calcination can be equal to or greater than the temperature in the oxidizing conditions used in the initial calcination, the optional cycle calcination, and the optional final calcination. In some embodiments, a sum of the time periods in the reduction conditions used in the initial calcination, the optional cycle calcination, and the optional final calcination can be greater than a sum of the time periods in the oxidizing conditions used in the initial calcination, the optional cycle calcination, and the optional final calcination.

The reduction conditions used in the initial calcination, the optional cycle calcination, and the optional final calcination can independently include heating the catalyst at a temperature in a range from 500° C., 525° C., 550° C., 575° C., 600° C., 625° C., 650° C., or 675° C. to 700° C., 725° C., 750° C., 775″C, 800° C., 825° C., or 850° C. The reduction conditions used in the initial calcination, the optional cycle calcination, and the optional final calcination independently include heating the catalyst for a time period in a range from 30 seconds, 1 minute, 5 minutes, 10 minutes, 15 minutes, 20 minutes, 25 minutes, or 30 minutes to 1 hour, 2 hours, 3 hours, 4 hours, 5 hours, 6 hours, 7 hours, 8 hours, 9 hours, or 10 hours, The reduction conditions used in the initial calcination, the optional cycle calcination, and the optional final calcination can independently include heating the catalyst under an absolute pressure in a range from 30 kPa, 60 kPa, or 90 kPa to 150 kPa, 300 kPa, or 600 kPa. It has been found by microscopic analysis that the incorporation of a reductive calcination. step in the process for calcining the synthesized catalyst as described herein was able to improve the distribution of the promoter (Sn) over the support when the promoter was used to make the synthesized catalyst.

It should be understood that a composition of the reducing vias, the temperature, and/or the pressure can be varied during any given calcination step, i.e., the initial calcination, the cycle calcination, and the final calcination. For example, if the initial calcination starts with the first reducing gas, the composition can start with a reducing gas that includes about 10 vol % of H₂ and can switch to a reducing gas that includes 100 vol % of H₂. Similarly, the initial calcination can start at a temperature of 550° C. for first duration and can increase to a temperature of 575° C. for a second duration of the initial calcination step. It should be understood that when the optional cycle calcination is used and n is ≥2, the composition of the second reducing gas, the temperature, time, and/or pressure used during each of the reduction conditions in the cycle calcination can be the same or different with respect to one another and can also vary during any give cycle calcination step.

The oxidizing conditions used in the initial calcination, the optional cycle calcination, and the optional final calcination can independently include heating the catalyst at a temperature in a range from 350° C., 375° C., 400° C., 425° C., 450° C., 475° C., 500° C., 525° C., 550° C. 575° C., or 600° C. to 625° C., 650° C. 675° C., 700° C., 725° C. 750° C., 775° C., 800° C. 825° C. or 850° C. The oxidizing conditions used in the initial calcination, the optional cycle calcination, and the optional final calcination can independently include heating the catalyst for a time period in a range from 30 seconds, 1 minute, 5 minutes, 10 minutes, 15 minutes, 20 minutes, 25 minutes, or 30 minutes to 1 hour, 2 hours, 3 hours, 4 hours, 5 hours, 6 hours, 7 hours, 8 hours, 9 hours, or 10 hours. The oxidizing conditions used in the initial calcination, the optional cycle calcination, and the optional final calcination can independently include heating the catalyst under an absolute pressure in a range from 30 kPa, 60 kPa, or 90 kPa to 150 kPa, 300 kPa, or 600 kPa.

It should be understood that a composition of the oxidizing gas, the temperature, and/or the pressure can be varied during any given calcination step, i.e., the initial calcination, the cycle calcination, and the final calcination. For example, if the initial calcination starts with the first oxidizing gas, the composition can start with an oxidizing gas that includes 10 vol % of O₂ and can switch to a reducing gas that includes 21 vol % of O₂, e.g., air. Similarly, the initial calcination can start at a temperature of 450° C. for first duration and can increase to a temperature of 475° C. for a second duration of the initial calcination step. It should be understood that when the optional cycle calcination is used and n is ≥2, the composition of the second oxidizing gas, the temperature, time, and/or pressure used during each of the oxidizing conditions in the cycle calcination can be the same or different with respect to one another and can also vary during any give cycle calcination step.

The first reducing gas, the second reducing gas, and the third reducing gas can independently be or include, but is not limited to, H₂, CO, CH₄, C₂H₆, C₃H₈, C₂H₄C₃H₆, steam, or any mixture thereof. In some embodiments, the first, second, and third reducing gas can independently be mixed with one or more inert gases. Suitable inert gases can be or can include, but are not limited to, He, Ne, Ar, N₂, CO₂, CH₄, or any mixture thereof. In some embodiments, a composition of the first, second, and third reducing gases can change or otherwise vary during the initial calcination, during the reduction conditions in the cycle calcination, and during the reduction conditions in the final calcination. For example, the initial calcination can start with a reducing gas that includes 100% H₂ and can switch to a reducing gas that includes 10% H₂ or any other amount of H₂ during the initial calcination. In other embodiments, the composition of the first, second, and third reducing gases can remain constant during the initial calcination, during the reduction conditions in the cycle calcination, and during the reduction conditions in the final calcination.

The first oxidizing gas, the second oxidizing gas, and the third oxidizing gas can independently be or include, but is not limited to, O₂, O₃, CO₂, steam, or any mixture thereof. In some embodiments, the first, second, and third oxidizing gas can independently be mixed with one or more inert gases. Suitable inert gases can be or can include, but are not limited to, He, Ne, Ar, N₂, CO₂, CH₄, or any mixture thereof In some embodiments, a composition of the first, second, and third oxidizing gases can change during the initial calcination, during the oxidizing conditions in the cycle calcination, and during the oxidizing conditions in the final calcination. For example, the initial calcination can start with a reducing gas that includes 100% and can switch to a reducing gas that includes about 21% O₂, e.g., air, or any other amount of O₂ during the initial calcination. In other embodiments, the composition of the first, second, and third oxidizing gases can remain constant during the initial calcination, during the oxidizing conditions in the cycle calcination, and during the oxidizing conditions in the final calcination.

Calcination on an industrial scale usually uses a box kiln, belt calciner, or rotary calciner. Calcination may also be aided by simultaneous microwave/ultrasonic treatments.

### Synthesized Catalyst

In some embodiments, the synthesized catalyst can include 0,001 wt %, 0,002 wt %, 0.003 wt %, 0,004 wt %, 0.005 wt %, 0.006 wt %, 0.007 wt %, 0.008 wt %, 0.009 wt %, 0.01 wt %, 0.015 wt %, 0.02 wt %, 0.025 wt %, 0.03 wt %, 0.035 wt %, 0.04 wt %, 0.045 wt %, 0.05 wt %, 0.055 wt %, 0.06 wt %, 0.065 wt %, 0.07 wt %, 0.075 wt %, 0.08 wt %, 0.085 wt %, 0.09 wt %, 0.095 wt %, 0.1 wt %, 0.2 wt %, 0.3 wt %, 0.4 wt %, 0,5 wt %, 0,6 wt %. 0.7 wt %, 0.8 wt %, 0.9 wt %, or 1 wt % to 2 wt %, 3 wt %, 4 wt %, 5 wt %, or 6 wt % of Pt disposed on a support, based on the non-volatile weight of the catalyst. In other embodiments, the synthesized catalyst can include ≤5.5 wt %, ≤4.5 wt %, ≤3.5 wt %, ≤2.5 wt %, ≤1.5 wt % ≤1 wt %, ≤0.9 wt %, ≤0.8 wt %, ≤0.7 wt %, ≤0.6 wt % ≤0.5 wt %, ≤0.4 wt %, ≤0.3 wt %, ≤0.2 wt %, ≤0.15 wt %, ≤0.1 wt %, ≤0.09 wt %, ≤0.08 wt %, ≤0.07 wt %, ≤0.06 wt %, ≤0.05 wt %, ≤0.045 wt %, ≤0.04 wt %, ≤0.035 wt %, ≤0.03 wt %, ≤0.025 wt %, ≤0.02 wt %, ≤0.015 wt %, ≤0.01 wt %, ≤0.009 wt %, ≤0.008 wt %, ≤0.007 wt %, ≤0.006 wt %, ≤0.005 wt %, ≤0.004 wt %, ≤0.003 wt %, ≤0.002, or ≤0.001 wt % of Pt disposed on the support, based on the non-volatile weight of the catalyst. In some embodiments, the synthesized catalyst can include >0.0001 wt %, >0.0005 wt %, >0.001 wt %, >0.003 wt %. >0.005 wt %, >0.007, >0.009 wt %, >0.01 wt %, >0.02 wt %, >0.04 wt %,>0.06 wt %, >0.08 wt %, >0.1 wt %, >0.13 wt %, >0.15 wt %, >0.17 wt %, >0.2 wt %, >0.2 wt %, >0.23, >0.25 wt %, >0.27 wt %, or >0.3 wt % and <0.5 wt %, <1 wt %, <2 wt %, <3 wt %, <4 wt %, <5 wt %, or <6 wt % of Pt disposed on the support, based on the non-volatile weight of the catalyst.

In some embodiments, the synthesized catalyst can optionally also include Ni, Pd, or a combination thereof, or a mixture thereof disposed on the support. If Ni, Pd, or a combination thereof, or a mixture thereof is also disposed on the support the synthesized catalyst can include 0.001 wt %, 0.002. wt %, 0.003 wt %, 0.004 wt %, 0.005 wt %, 0.006 wt %, 0.007 wt %, 0.008 wt %, 0.009 wt %, 0.01 wt %, 0.015 wt %, 0.02 wt %, 0.025 wt %, 0.03 wt %, 0.035 wt %, 0.04 wt %, 0.045 wt %, 0.05 wt %, 0.055 wt %, 0.06 wt %, 0.065 wt %, 0.07 wt %, 0.075 wt %, 0.08 wt %, 0.085 wt %, 0.09 wt %, 0.095 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, 5 wt %, or 6 wt % of a combined amount of Pt and any Ni and/or any Pd disposed on the support, based on the non-volatile weight of the catalyst. In some embodiments, an active component of the synthesized catalyst that can be capable of effecting one or more of reforming or dehydrogenation, dehydroaromatization, and dehydrocyclization of a hydrocarbon-containing feed can include the Pt or the Pt and Ni and/or Pd, It should be understood that the active component may not be active or may be less active as compared to the calcined catalyst obtained at the end of the cycle calcination or at the end of the final calcination. It should also be understood that the Pt and, if present, Ni and/or Pd, can be present in the elemental form and/or in the form of a compound containing Pt, and if present, a compound containing Ni and/or a compound containing Pd in the synthesized catalyst.

In some embodiments, the synthesized catalyst can include a promoter in an amount of up to 10 wt % disposed on the support, based on the non-volatile weight of the catalyst. The promoter can be or can include, but is not limited to, Sn, Cu, Au. Ag. Ga, or a combination thereof, or a mixture thereof. In some embodiments, the promoter can be associated with the Pt and/or, if present, the Ni and/or Pd. For example, the promoter and the Pt disposed on the support can form Pt-promoter clusters that can be dispersed on the support. The promoter can improve the selectivitylactivity/longevity of the catalyst for a given upgraded hydrocarbon. In some embodiments, the promoter can improve the propylene selectivity of the catalyst when the hydrocarbon-containing feed includes propane. The synthesized catalyst can include the promoter in an amount of 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 3 wt %, 5 wt %, 7 wt %, or 10 wt %, based on the non-volatile weight of the catalyst. It should be understood that the promoter may not be associated with or may be less associated with the Pt and/or, if present, the Ni and/or Pd, as compared to the calcined catalyst obtained at the end of the cycle calcination or at the end of the final calcination. It should also be understood that the promoter can be present in the elemental form and/or in the form of a compound containing the promoter in the synthesized catalyst.

In some embodiments, the synthesized catalyst can optionally include one or more alkali metal elements in an amount of up to 5 wt % disposed on the support, based on the non-volatile weight of the catalyst. The alkali metal element, if present, can be or can include, but is not limited to, Li, Na, K. Rb, Cs, or a combination thereof, or a mixture thereof In at least some embodiments, the alkali metal element ca be or can include K and/or Cs. In some embodiments, the alkali metal element, if present, can improve the selectivity of the catalyst particles for a given upgraded hydrocarbon. The synthesized catalyst can include the alkali metal element in an amount of 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, or 5 wt %, based on the non-volatile weight of the catalyst. It should be understood that the alkali metal element(s), if present, can be in the elemental form andlor in the form of a compound(s) containing the alkali metal element(s).

The support can be or can include, but is not limited to, one or more Group 2 elements, or a combination thereof, or a mixture thereof. In some embodiments, the Group 2 element can be present in its elemental form. In other embodiments, the Group 2 element can be present in the form of a compound. For example, the Group 2 element can be present as an oxide, a phosphate, a halide, a 14alite, a sulfate, a sulfide, a borate, a nitride, a carbide, an illuminate, an alwninosilicate, a silicate, a carbonate, metaphosphate, a selenide, a tungstate, a molybdate, a chromite, a chromate, a dichromate, or a silicide. In some embodiments, a mixture of any two or more compounds that include the Group 2 element can be present in different forms. For example, a first compound can be an oxide and a second compound can be an alwninate where the first compound and the second compound include the same or different Group 2 element, with respect to one another.

The synthesized catalyst can include ≥0.5 wt %, ≥1 wt %, ≥2 wt %, >3 wt %, ≥4 wt %, ≥5 wt %, ≥6 wt %, ≥7 wt %, ≥8 wt %, ≥9 wt %, ≥10 wt %, ≥11 wt %, ≥12 wt % 0, ≥13 wt %, ≥14 wt %, ≥15 wt %, ≤16 %, ≥17 wt %, ≥18 wt %, ≥19 wt %, ≥20 wt %, ≥21 wt %, ≥22 wt %, ≥23 wt %, ≥24 wt %, ≥25 %, ≥26 wt %, ≥27 wt %, ≥28 wt %, ≥29 wt %, ≥30 wt %, ≥35 wt %, ≥40 wt %, ≥45 wt %, ≥50 wt %, ≥55 wt %, ≥60 wt %, ≥65 wt %, ≥70 wt %, ≥75 wt %, ≥80 wt %, ≥85 wt %, or ≥90 wt % of the Group 2 element, based on the non-volatile weight of the catalyst. In some embodiments, the synthesized catalyst can include the Group 2 element in a range of from 0.5 wt %, 1 wt %, 2 wt %, 2.5 wt %, 3 wt %, 5 wt %, 7 wt %, 10 wt %, 11 wt %, 13 wt %, 15 wt %, 17 wt %, 19 wt %, 21 wt %, 23 wt %, or 25 wt % to 30 wt %, 35 wt %, 40 wt %, 45 wt %, 50 wt %, 55 wt %, 60 wt %, 65 wt, 70 wt %, 75 wt %, 80 wt %, 85 wt %, 90 wt %, or 92.34 wt %, based on the non-volatile weight of the catalyst. In some embodiments, a molar ratio of the Group 2 element to the Pt or the Pt and any Ni andlor Pd present can be in a range from 0.24, 0.5. 1, 10, 50, 100, 300, 450, 600, 800, 1,000, 1,200, 1,500, 1,700, or 2,000 to 3,000, 3,500, 4,000, 4,500, 5,000, 5,500, 6,000, 6,500, 7,000, 7,500, 8,000, 8,500, 9,000, 9,500, 10,000 ,15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 200,000, 300,000, 400,000, 500,000, 600,000, 700,000, 800,000, or 900,000.

In some embodiments, the support can include the Group 2 element and Al and can be in the form of a mixed Group 2 element/Al metal oxide that has O, Mg, and Al atoms mixed on an atomic scale. In some embodiments the support can be or can include the Group 2 element and Al in the form of an oxide or one or more oxides of the Group 2 element and Al₂O₃ that can be mixed on a nm scale. In some embodiments, the support can be or can include an oxide of the Group 2 element, e.g., MgO, and Al₂O₃ mixed on a am scale.

In some embodiments, the support can be or can include a first quantity of the Group 2 element and Al in the form of a mixed Group 2 element/Al metal oxide and a second quantity of the Group 2 element in the form of an oxide of the Group 2 element. In such embodiment, the mixed Group 2 element/Al metal oxide and the oxide of the Group 2 element can be mixed on the nm scale and the Group 2 element and Al in the mixed Group 2 element/Al metal oxide can be mixed on the atomic scale.

In other embodiments, the support can be or can include a first quantity of the Group 2 element and a first quantity of Al in the form of a mixed Group 2 element/AI metal oxide, a second quantity of the Group 2 element in the form of an oxide of the Group 2 element, and a second quantity of Al in the form of Al₂O₃. In such embodiment, the mixed Group 2 element/Al metal oxide, the oxide of the Group 2 element, and the Al₂O₃ can be mixed on a nm scale and the Group 2 element and Al in the mixed Group 2 element/Al metal oxide can be mixed on the atomic scale.

In some embodiments, when the support includes the Group 2 element and Al, a weight ratio of the Group 2 element to the Al in the support can be in a range from 0.001, 0.005, 0.05, 0,1, 0.15, 0,2, 0.3, 0.5, 0.7, or 1 to 3, 6, 12.5, 25, 50, 75. 100, 200, 300, 400, 500, 600, 700, 800, 900, or 1,000. In some embodiments, when the support includes Al, the synthesized catalyst can include Al in a range from 0.5 wt %, 1 wt %, 1.5 wt %, 2 wt %, 2.1 wt %, 2.3 wt %, 2.5 wt %, 2.7 wt %, 3 wt %, 4 wt %, 5 wt %, 6 wt %, 7 wt %, 8 wt %, wt %, 10 wt %,or 11 wt % to 15 wt %, 20 wt %, 25 wt %, 30 wt %, 40 wt %, 45 wt %, or 50 wt %, based on the non.-volatile weight of the catalyst.

In some embodiments, the support can be or can include, but is not limited to, one or more of the following compounds: Mg_{w}Al₂O_{3+w}, where w is a positive number, CaₓAl₂O₃₊ₓ, where x is a positive number; Sr_{y}Al₂O_{3+y}, where y is a positive number; Ba_{z}Al₂O_{3+z}, where z is a positive number. BeO; MgO; CaO; BaO; SrO; BeCO₃; MgCO₃; CaCO₃; SrCO₃, BaCO₃; CaZrO₃; Ca₇ZrAl₆O₁₈; CaTiO₃; Ca₇Al₆O₁₈; Ca₇HfAl₆O₁₈; BaCeO₃; one or more magnesium chromates, one or more magnesium tungstates, one or more magnesium molybdates, combinations thereof, and mixtures thereof. In some embodiments, the Group 2 element can include Mg and at least a portion of the Group 2 element can be in the form of MgO or a mixed oxide that includes MgO. In some embodiments, the support can be or can include, but is not limited to, a MgO-Al₂O₃ mixed metal oxide, In some embodiments, when the support is a IMO-Al₂O₃ mixed metal oxide, the support can have a molar ratio of Mg to Al equal to 20, 10, 2, 1 to 0.5, 0.1, or 0.01.

The Mg_{w}Al₂O_{3+w}, where w is a positive number, if present as the support or as a component of the support can have a molar ratio of Mg to Al in a range from 0.5. 1, 2, 3, 4, or 5 to 6, 7, 8, 9, or 10. In some embodiments, the Mg_{w}Al₂O_{3+w} can include MgAl₂O₄, Mg2Al2O5, or a mixture thereof. The CaₓAl₂O₃₊ₓ, where x is a positive number, if present as the support or as a component of the inorganic support can have a molar ratio of Ca to Al in a range from 1:12, 1:4, 1:2, 2:3, 5:6, 1:1, 12:14, or 1.5:1. In some embodiments, the Caₓ,Al₂O₃₊ₓ can include tricalcium aluminate, dodecacalciurn hepta-aluminate, monocalciurn aluminate, monocalcium 16alite16nate, monocalcium hexa-aluminate, dicalcium aluminate, pentacalcium trialuminate, tetracalcium trialuminate, or any mixture thereof. The Sr_{y}Al₂O_{3+y}, where y is a positive number, if present as the support or as a component of the support can have a molar ratio of Sr to Al in a range from 0.05, 0.3, or 0.6 to 0.9, 1.5, or 3. The Ba_{z}Al₂O_{3+z}, where z is a positive number, if present as the support or as a component of the support can have a molar ratio of Ba to Al 0.05, 0.3, or 0.6 to 0.9, 1.5, or 3.

In some embodiments, the support can also include, but is not limited to, at least one metal element and/or at least one metalloid element selected from Groups other than Group 2 and Group 10 and/or at least one compound thereof, where the at least one metal element and/or at least one metalloid element is not one of the alkali metal elements or one of the promoter elements. If the support also includes a compound that includes the metal element and/or metalloid element selected from Groups other than Group 2 and Group 10, where the at least one metal element and/or at least one metalloid element is not one of the alkali metal elements or one of the promoter elements, the compound can be present in the support as an oxide, a phosphate, a halide, a 16alite, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide. In some embodiments, the at least one metal element and/or at least one metalloid element selected from Groups other than Group 2 and Group 10 and/or at least one compound thereof, where the at least one metal element and/or at least one metalloid element is not one of the alkali metal elements or one of the promoter elements, can be or can include, but is not limited to, one or more rare earth elements, i.e., elements having an atomic number of 21, 39, or 57 to 71.

If the support includes the at least one metal element and; or at least one metalloid element selected from Groups other than Group 2 and Group 10 and/or at least one compound thereof, where the at least one metal element and/or at least one metalloid element is not one of the alkali metal elements or one of the promoter elements, the at least one metal element and/or at least one metalloid element can, in some embodiments, function as a hinder and can be referred to as a “binder”. Regardless of whether or not the at least one metal element and/or at least one metalloid element selected from Groups other than Group 2 and Group 10 and/or at least one compound thereof, where the at least one metal element and/or at least one metalloid element is not one of the alkali metal elements or one of the promoter elements, the at least one metal element and/or at least one metalloid element selected from Groups other than Group 2 and Group 10 will be further described herein as a “binder” for clarity and ease of description. In some embodiments, when the support includes the binder, the synthesized catalyst can include the binder in a range of from 0.01 wt %, 0.05 wt %, 0.1 wt %, 0.5 wt %, 1 wt %, 5 wt %, 10 wt %, 15 wt %, 20 wt %, 25 wt %, 30 wt %, 35 wt % or 40 wt % to 50 wt %, 60 wt %, 70 wt %, 80 wt %, or 90 wt %, based on the non-volatile weight of the catalyst.

In some embodiments, suitable compounds that include the binder can be or can include, but are not limited to, one or more of the following: B₂O₃, AlBO₃, Al₂O₃, SiO₂, ZrO₂, TiO₂, SiC, Si₃N₄, an aluminosilicate, zinc aluminate, ZnO, VO, VO₂, V₂O₅, Ga_{g}Oₜ, InᵤO_{y}, Mn₂O₃, Mn₃O₄, MnO, one or more molybdenum oxides, one or more tungsten oxides, one or more zeolites, where s, t, u, and v are positive numbers and mixtures and combinations thereof.

In some embodiments, the synthesized catalyst can be in the form of monolithic structures. In other embodiments, the synthesized catalyst can be in the form of particles. In some embodiments, the synthesized catalyst particles can have a median particle size in a range from 1 μm, 5 μm, 10 μm, 20 μm, 40 μm, or 60 μm to 80 μm, 100 μm, 115 μm, 130 μm, 150 μm, 200 μm, 300 μm or 400, or 500 μm. In sonic embodiments, the synthesized catalyst particles can have an apparent loose bulk density in a range from 0.3 g/cm³, 0.4 g/cm³, 0.5 g/cm³, 0.6 g/cm³, 0.7 g/cm³, 0.8 g/cm³, 0.9 g/cm³, or 1 g/cm³ to 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, or 2 g/cm³, as measured according to ASTM D7481-18 modified with a 10, 25, or 50 mL graduated cylinder instead of a 100 or 250 mL graduated cylinder. in some embodiments, the synthesized catalyst particles can have an attrition loss after one hour of ≤5 wt %, ≤4 wt %, ≤3 wt %, ≤2 wt %, ≤1 wt %, ≤0.7 wt %, ≤0.5 wt %, ≤0.4 wt %, ≤0.3 wt %, ≤0.2 wt %, ≤0.1 wt %, ≤0.07 wt %, or ≤0.05 wt %, as measured according to ASTM D5757-11(2017). The morphology of the synthesized catalyst particles is largely spherical so that they are suitable to run in a fluid bed reactor. In some embodiments, the synthesized catalyst particles can have a size and density that is consistent with a Geldart A or Geldan B definition of a fluidizable solid.

In some embodiments, the synthesized catalyst particles can have a surface area in a range from 0.1 m²/g, 1m²/g, 10 m²/g, or 100 m²/g to 500 m²/g, 800 m²/g, 1,000 m²/g, or 1,500 m²/g. The surface area of the synthesized catalyst particles can be measured according to the Brunauer-Emmett-Teller (BET) method using adsorption-desoiption of nitrogen (temperature of liquid nitrogen, 77 K) with a Micromeritics 3flex instrument after degassing of the powders for 4 hrs at 350° C. More information regarding the method can be found, for example, in “Characterization of Porous Solids and Powders: Surface Area, Pore Size and Density,” S. Lowell et al., Springer, 2004.

### Process for Making the Synthesized Catalyst

The process for making the synthesized catalyst can include preparing a slurry or gel that can include, milling, mixing, blending, combining, or otherwise contacting, but is not limited to, a compound containing a Group 2 element and a liquid medium. In some embodiments, preparation of the slurry or gel can also include contacting, but is not limited to, the compound containing the Group 2 element, the liquid medium, and one or more additives. In other embodiments, the preparing the slurry or gel can include contacting, but is not limited to, the compound containing the Group 2 element, the liquid mediwn, a binder or binder precursor, and, optionally, one or more additives.

The compound containing the Group 2 element can be in the form of an oxide, a hydroxide, a hydrated carbonate, a salt, a clay containing a Group 2 element, a layered double hydroxide, a phosphate, a halide, a halate, a sulfate, a sulfide, a borate, a nitride, a carbide, an aluminate, an aluminosilicate, a silicate, a carbonate, metaphosphate, a selenide, a tungstate, molybdate, a chromite, a chromate, a dichromate, a silicide, or a mixture thereof. In some embodiments, the Group 2 element can be or can include Mg and the compound containing the Group 2 element can be in the form of a magnesium oxide, a magnesium hydroxide, hydromannesite (a hydrated magnesium carbonate mineral, Mg₅(CO₃)₄(OH)₂*4H₂O), a magnesiwn salt, a magnesium-containing clay, hydrotalcite (a layered double hydroxide), an organo-magnesium compound or a mixture thereof In some embodiments, the Group 2 element can be or can include Mg and the compound containing the Group 2 element can be in the form of a calcined magnesium oxide, a calcined magnesium hydroxide, calcined hydromagnesite (a hydrated magnesium carbonate mineral, Mg₅(CO₃)₄(OH)₂* 4H₂O), a calcined magnesium salt, a calcined magnesium-containing clay, calcined hydrotal cite (a layered double hydroxide), a calcined organo-magnesium compound, or a mixture thereof.

The liquid medium can be or can include, but is not limited to, water, alcohols, acetone, chloroform, methylene chloride, dimethyl formamide, dimethyl sulfoxide, glycerin, ethyl acetate, or any mixture thereof. Illustrative alcohols can be or can include, but are not limited to methanol, ethanol, isopropanol, or any mixture thereof. The binder, if present, can be or can include the binders described above. The binder precursor, if present, can be or can include, but is not limited to, Al₂Si₂O₅(OH)₄ (Kaolin clay), aluminum chlorohydrol, boehmite, pseudoboehmite, gibbsite, bayerite, aluminum nitrate, aluminum chloride, sodium aluminate, alumina sol, silica sol, or any mixture thereof. It is known that in literature, some of the compounds herein referred to as “binders” may also be referred to as fillers, a matrix, an additive, etc. The one or more additives, if present, can be or can include, but is not limited to, acids such as formic acid, lactic acid, citric acid, acetic acid, HNO₃, HCl, oxalic acid, stearic acid, carbonic acid, etc.; bases such as ammonia solution, NaOH, KOH, etc.; inorganic salts such as nitrates, carbonates, bicarbonates, chlorides, etc.; organic salts such as acetates, oxalates, formates, citrates, etc.; polymers such as polyvinyl alcohol, polysaccharide, etc., or any mixture thereof. The additives can belp to improve the chemical/physical property of the spray dried material and/or to improve the rheological property of the slimy/gel to facilitate spray drying.

The slimy or gel can be spray dried to produce spray dried support particles that include the Group 2 element. Spray drying refers to the process of producing a dry particulate solid product from the slurry or the gel. The process can include spraying or atomizing the slurry or gel, e.g., forming small droplets, into a temperature-controlled gas stream to evaporate the liquid medium from the atomized droplets and produce the particulate solid product. For example, in the spray drying process, the slurry or gel can be atomized to small droplets and mixed with hot air or a hot inert gas, e.g., nitrogen, to evaporate the liquid from the droplets. The temperature of the slurry or gel during the spray drying process can usually be close to or greater than the boiling temperature of the liquid. An outlet air temperature of about 60° C. to about 120° C. can be common.

The slurry or gel can be atomized with one or more pressure nozzles (e.g., a fluid nozzle atomizer), one or more pulse atomizers, one or more high speed spinning discs (e.g., centrifugal or rotary atomizer), or any other known process. The median particle size, liquid (e.g., water) concentration, apparent loose bulk density, or any combination thereof, of the particulate solid product prepared via spray drying can be controlled, adjusted, or otherwise influenced by one or more operating conditions and/or parameters of the spray dryer. Illustrative operating conditions can include, but are not limited to, the feed rate and temperature of the gas stream, the atomizer velocity, the feed rate of the slurry or gel via the atomizer, the temperature of the. slurry or gel, the size and/or solids concentration of the droplets, the spray dryer dimensions, or any combination thereof. It is well-known in the art that the various operating conditions will vary depending on the particular spray drying apparatus that is used and can be readily determined by persons having ordinary skill in the art.

In some embodiments, the spray dried support particles can be calcined under an oxidative atmosphere, e.g., air, to produce calcined support particles that include the Group 2 element. In some embodiments, the spray dried support particles can be calcined at a temperature in a range of from 450° C., 500° C., 525° C., 550° C., 575° C., 600° C., 625° C., 650° C., or 675° C. to 700° C., 725° C., 750° C., 775° C., 800° C., 850° C., 900° C., 950° C., or more. In some embodiments, the spray dried support particles can be calcined at a temperature of ≤950° C., ≤900° C., ≤850° C., ≤800° C. ≤750° C. ≤700° C., ≤650° C., ≤600° C., or ≤550° C. ≤525° C. ≤500° C. ≤475° C., or ≤460° C. In some embodiments, the spray dried support particles can be calcined for a time period of ≤240 minutes ≤180 minutes ≤120 minutes ≤90 minutes, ≤60 minutes, ≤45 minutes, ≤30 minutes, ≤25 minutes, ≤20 minutes, or ≤15 minutes. In some embodiments, the spray dried support particles can be calcined in the presence of oxygen, e.g,, air. In some embodiments, the spray dried particles can be calcined at a temperature in a range of from 550° C. to 900° C. or 550° C. to 850° C. for a time period of ≤240 minutes ≤180 minutes ≤120 minutes ≤90 minutes, ≤60 minutes, ≤45 minutes, ≤30 minutes, ≤25 minutes, ≤20 minutes, or ≤15 minutes. In other embodiments, the spray dried particles are calcined at a temperature of ≤550° C., ≤540° C., ≤530° C., ≤520° C., ≤510° C., or ≤500° C. for a time period of <240 minutes ≤180 minutes ≤120 minutes ≤90 minutes, ≤60 minutes, ≤45 minutes, ≤30 minutes, minutes, ≤25 minutes, or ≤15 minutes.

The Pt and, if present, Ni and/or Pd, present in the synthesized catalyst can be introduced via one or two ways. For simplicity, Pt will be described, but in addition to the Pt, a Ni-containing and/or Pd-containing compound could also be used. In some embodiments, the process for making the synthesized catalyst can include (i) contacting at least the compound containing the Group 2 element and the liquid medium with a Pt-containing compound such that the Pt can be present in the slurry or the gel and the synthesized catalyst can include spray dried catalyst particles that include the support particles having Pt disposed thereon. In such embodiment, the spray dried particles can be the synthesized catalyst or the spray dried particles could be subjected to equilibration and/or drying, but not at a temperature of ≥350° C. to produce the synthesized catalyst.

In other embodiments, the process for making the synthesized catalyst can include (ii) depositing Pt on the calcined spray dried particles by contacting the calcined spray dried particles with a Pt-containing compound to produce Pt-containing calcined spray dried particles. In some embodiments, the calcined spray dried particles can be contacted with the Pt-containing compound in the presence of a liquid medium to produce a mixture and the solid fraction can be recovered by filtration. The Pt-containing compound can be or can include, but is not limited to, chloroplatinic acid hexahydrate, tetraammineplatinum(II) nitrate, platinum(II) acetylacetonate, platinum(II) bromide, platiniun(II) iodide, platinum(II) chloride, platinum(IV) chloride, platinum(II)diammine de, ammonium tetrachl oroplatinate(II), tetraammineplatinurn(II) chloride hydrate, tetraammineplatinum(II) hydroxide hydrate, or any mixture thereof Suitable Ni- and Pd-containing compounds can be or can include, but are not limited to, nickel (II) chloride, pailadium(ll) acetate, pal ladi um(II) nitrate, or a mixture thereof.

The promoter, and/or alkali metal element that can optionally be present in the synthesized catalyst can be introduced in the same way as the Pt can be introduced. The compound that includes the promoter element can be or can include, but is not limited to, tin(II) oxide, tin(IV) oxide, tin(IV) chloride pentahydrate, tin(II) chloride dihydrate, tin(II) bromide, tin(IV) bromide, tin(II) acetylacetonate, tin(II) acetate, tin(IV) acetate, silver(I) nitrate, gold(III) nitrate, copper(II) nitrate, galliutn(an) nitrate, or any mixture thereof. The compound that includes the alkali metal element can be or can include, but is not limited to, lithium nitrate, sodium nitrate, potassium nitrate, rubidium nitrate, cesium nitrate, or any mixture thereof.

In some embodiments, platinum UI) oxalate and tin(II) oxalate can be used as the Pt-containing compound and the Sn-containing compound. Tin(II) oxalate can be dissolved in an aqueous solution containing ammonium oxalate or an aqueous solution containing ammonium oxalate and platinum oxalate. The aqueous solution containing tin(II) oxalate and ammonium oxalate or ammonium oxalate and platinwn oxalate can be added to the support, followed by equilibration, drying, and/or calcination. The Sn distribution across the support can be improved by using oxalates of Sn including tin(II) oxalate and tin(IV) oxalate as the Sn-containing compounds. The use of platinum (II) oxalate and tin(II) oxalate as the Pt-containing compound and the Sn-containing compound on a different support for a different application has been described in U.S. Pat. No. 8,569,20382.

### A First Process for Upgrading a Hydrocarbon

The first process for upgrading a hydrocarbon can include contacting a first hydrocarbon-containing feed with the calcined catalyst to effect one or more of dehydrogenation, dehydroaromatization, and dehydrocyclization of at least a portion of the first hydrocarbon-containing feed to produce a coked catalyst and an effluent that can include one or more upgraded hydrocarbons and molecular hydrogen. The calcined catalyst and the first hydrocarbon-containing feed can be contacted with one another within any suitable environment such as one or more reaction or conversion zones disposed within one or more reactors to produce the effluent and the coked catalyst. The reaction or conversion zone can be disposed or otherwise located within one or more fixed bed reactors, one or more fluidized or moving bed reactors, one or more reverse flow reactors, or any combination thereof.

The first hydrocarbon-containing teed and calcined catalyst can be contacted at a temperature in a range from 300° C., 350° C., 400° C., 450° C., 500° C., 550° C., 600° C., 620° C., 650° C., 660° C., 670° C., 680° C., 690° C., or 700° C., to 725° C., 750° C., 760° C., 780° C., 800° C., 825° C., 850° C., 875° C., or 900° C. In some embodiments, the first hydrocarbon-containing feed and the calcined catalyst can be contacted at a temperature of at least 620° C., at least 650° C., at least 660° C., at least 670° C., at least 680° C., at least 690° C., or at least 700° C. to 725° C., 750° C., 760° C., 780° C., 800° C., 825° C., 850° C., 875° C., or 900° C. The first hydrocarbon-containing feed can be introduced into the reaction or conversion zone and contacted with the calcined catalyst therein for a time period of ≤3 hours, ≤2.5 hours, ≤2 hours, ≤1.5 hours, ≤1 hour, ≤45 minutes, ≤30 minutes, ≤20 minutes, ≤10 minutes, ≤5 minutes, ≤1 minute, ≤30 seconds, ≤10 seconds, ≤5 seconds, or ≤1 second or ≤0.5 second. In some embodiments, the first hydrocarbon-containing feed can be contacted with the calcined catalyst for a time period in a range from 0.1 seconds, seconds, 0.7 seconds, I second, 30 second, 1 minute, 5 minutes, or 10 minutes to 30 minutes, minutes, 70 minutes, 1.5 hours, 2 hours, or 3 hours.

The first hydrocarbon-containing feed and the calcined catalyst can be contacted under a hydrocarbon partial pressure of at least 20 kPa-absolute, where the hydrocarbon partial pressure is the total partial pressure of any C₂-C₁₆ alkanes and any C₈-C₁₆ alkyl_{—} aromatics in the first hydrocarbon-containing feed. In some embodiments, the hydrocarbon partial pressure during contact of the first hydrocarbon-containing feed and the calcined catalyst can be in a range from 20 kPa-absolute, 50 kPa-absolute, 100 kPa-absolute, at least 150 kPa, at least 200 kPa 300 kPa-absolute, 500 kPa-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute, where the hydrocarbon partial pressure is the total partial pressure of any C₂-C₁₆ alkanes and any C₈-C₁₆ alkyl aromatics in the first hydrocarbon-containing feed. In other embodiments, the hydrocarbon partial pressure during contact of the hydrocarbon-containing feed and the calcined catalyst can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute, where the hydrocarbon partial pressure is the total partial pressure of any C₂-C₁₆ alkanes and any C₈-C₁₆ alkyl aromatics in the first hydrocarbon-containing feed.

In some embodiments, the first hydrocarbon-containing feed can include at least 60 vol %, at least 65 vol %, at least 70 vol %, at least 75 vol %, at least 80 vol %, at least 85 vol %, at least 90 vol %, at least 95 vol %, or at least 99 vol % of a single C₂-C₁₆ alkane, e.g., propane, based on a total volume of the first hydrocarbon-containing feed. The first hydrocarbon-containing feed and calcined catalyst can be contacted under a single C₂-C₁₆ alkane, e.g., propane, pressure of at least 20 kPa-absolute, at Least 50 kPa-absolute, at least 100 kPa-absolute, at least 150 kPa-absolute, at least 250 kPa-absolute, at least 300 kPa-absolute, at least 400 kPa-absolute, at least 500 kPa-absolute, or at least 1,000 kPa-absolute.

The first hydrocarbon-containing feed can be contacted with the calcined catalyst within the reaction or conversion zone at any weight hourly space velocity (WHSV) effective for carrying out the upgrading process. In some embodiments, the WHSV can be 0.01 hr⁻¹, 0.1 hr⁻¹, 1 hr⁻¹, 2 hr⁻¹, 5 hr^{31 1}, 10 hr⁻¹, 20 hr^{31 1}, 30 hr⁻¹, or 50 hr⁻¹ to 100 hr⁻¹, 250 hr⁻¹, 500 hr⁻¹, or 1,000 hr⁻¹. In some embodiments, when the hydrocarbon upgrading process includes a fluidized or otherwise moving calcined catalyst, a ratio of the calcined catalyst circulation mass flow rate to a combined amount of any C₂-C₁₆ alkanes and any C₈-C₁₆ alkyl aromatics mass flow rate can be in a range from 1, 3, 5, 10, 15, 20, 25, 30, or 40 to 50, 60, 70, 80, 90, 100, 110, 125, or 150 on a weight to weight basis.

When the activity of the coked catalyst decreases below a desired minimum amount, the coked catalyst or at least a portion thereof can be subjected to a regeneration process to produce a regenerated catalyst. More particularly, the coked catalyst can be contacted with one or more oxidants to effect combustion of at least a portion of the coke to produce a regenerated catalyst lean in coke and a combustion gas. Regeneration of the coked catalyst can occur within the reaction or conversion zone or within a combustion zone that is separate and apart from the reaction or conversion zone, depending on the particular reactor configuration, to produce a regenerated catalyst. For example, regeneration of the coked catalyst can occur within the reaction or conversion zone when a fixed bed or reverse flow reactor is used, or within a separate combustion zone that can be separate and apart from the reaction or conversion zone when a fluidized bed reactor or other circulating or fluidized type reactor is used. In some embodiments, fuel may be added to the combustion zone to generate heat that can beat up the coked catalysts. Illustrative fuels can be or can include, but are not limited to, hydrocarbons, e.g., methane, ethane, propane, butane, pentane, or hydrocarbon containing streams, e.g., natural gas, molecular hydrogen, fuel oil, heavy fuel oil, gasoline, diesel, kerosene, distillate, and/or other combustible compounds. In some embodiments, the regeneration process can include burning fuels in the combusting zone, followed by flowing relatively dry oxidant(s) through the combustion zone to produce the regenerated catalyst. In some embodiments, the regeneration process can include burning fuels in a first combustion zone with the coked catalyst to produce an at least partially regenerated catalyst, transporting the at least partially regenerated catalyst to a second combustion zone, and flowing relatively dry oxidant(s) through second combustion zone to produce the regenerated catalyst. An example of a dry oxidant includes air that contains <2 vol % of water vapor.

In some embodiments the process can optionally include contacting at least a portion of the regenerated catalyst with a reducing gas to produce a regenerated and reduced catalyst. An additional quantity of the hydrocarbon-containing feed can be contacted with at least a portion of the regenerated catalyst and/or at least a portion of any regenerated and reduced catalyst to produce a re-coked catalyst and additional effluent.

In some embodiments, a cycle time from contacting the hydrocarbon-containing feed with the calcined catalyst to contacting the additional quantity of the hydrocarbon-containing feed with the regenerated catalyst can be ≤5 hours. The first cycle begins upon contact of the calcined catalyst with the first hydrocarbon-containing feed, followed by contact with at least the oxidative gas to produce the regenerated catalyst or at least the oxidative gas and the optional reducing gas to produce the regenerated and reduced catalyst, and the first cycle ends upon contact of the regenerated catalyst with the additional quantity of the first hydrocarbon-containing feed. if one or more additional feeds (described in more detail below) are utilized between flows of the first hydrocarbon-containing feed and the oxidative gas, between the oxidative gas and the reducing gas (if used), between the oxidative gas and the additional quantity of the first hydrocarbon-containing feed, anchor between the reducing gas (if used) and the additional quantity of the first hydrocarbon-containing feed, the period of time such stripping gas(es) is/are utilized would be included in the period included in the cycle time. As such, the cycle time from contacting the first hydrocarbon-containing feed with the calcined catalyst in to the contacting the additional quantity of the hydrocarbon-containing feed with the regenerated catalyst, in some embodiments, can be ≤5 hours, ≤4 hours, ≤3 hours, ≤2 hours, ≤1 hour, ≤55 minutes, ≤50 minutes, or ≤45 minutes.

The oxidant can be or can include, but is not limited to, O₂, O₃, CO₂, H₂O, or a mixture thereof. In some embodiments, an amount of oxidant in excess of that needed to combust 100% of the coke on the coked catalyst can be used to increase the rate of coke removal from the catalyst, so that the time needed for coke removal can be reduced and lead to an increased yield in the upgraded product produced within a given period of time. The use of pure O₂ as an oxidant can facilitate the capturing and sequestration of CO₂ made during combustion in one or more downstream CO₂ recovery systems.

The coked catalyst and oxidant can be contacted with one another at a temperature in a range from 500° C., 550° C., 600° C., 650° C., 700° C., 750° C. or 800° C. to 900° C., 950° C., 1.000° C., 1,050° C., or 1,100° C. to produce the regenerated catalyst. In some embodiments, the coked catalyst and oxidant can be contacted with one another at a temperature in a range from 500° C. to 1,100° C., 600° C. to 1,000° C., 650° C. to 950° C., 700° C. to 900° C., or 750° C. to 850° C. to produce the regenerated catalyst.

The coked catalyst and oxidant can be contacted with one another for a time period of ≤2 hours, ≤1 hour, ≤30 minutes, ≤10 minutes, ≤5 minutes, ≤1 min, ≤30 seconds, ≤10 seconds, ≤5 seconds, or ≤1 second. For example, the coked catalyst and oxidant can be contacted with one another for a time period in a range from 2 seconds to 2 hours. In some embodiments, the coked catalyst and oxidant can be contacted for a time period sufficient to remove ≥50 wt %, ≥75 wt %, or ≥90 wt % or ≥99% of any coke disposed on the coked catalyst.

In some embodiments, the time period the coked catalyst and oxidant contact one another can be less than the time period the calcined/regenerated catalyst contacts the hydrocarbon-containing feed to produce the effluent and the coked catalyst. For example, the time period the coked catalyst and oxidant contact one another can be at least 90%, at least 60%, at least 30%, or at least 10% less than the time period the calcinedlregenerated catalyst contacts the hydrocarbon-con.taining feed to produce the effluent. In other embodiments, the time period the coked catalyst and oxidant contact one another can be greater than the time period the cal cined/regenera.ted catalyst contacts the hydrocarbon-containing feed to produce the effluent and the coked catalyst. For example, the coked catalyst and oxidant contact one another can be at least 50%, at least 100%, at least 300%, at least 500%, at least 1,000%, at least 10,000%, at least 30,000%, at least 50,000%, at least 75,000%, at least 100,000%, at least 250,000%, at least 500,000%, at least 750,000%, at least 1,000,000%, at least 1,250,000%, at least 1,500,000%, or at least 1,800,000% greater than the time period the calcined/regenerated catalyst contacts the hydrocarbon-containing feed to produce the effluent.

The coked catalyst and oxidant can be contacted with one another under an oxidant partial pressure in a range from 20 kPa-absolute, 50 kPa-absolute, 100 kPa-absolute, 300 kPa-absolute, 500 kPa-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute. In other embodiments, the oxidant partial pressure during contact with the coked catalyst can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the regenerated catalyst.

Without wishing to be bound by theory, it is believed that at least a portion of the Pt and, if present, and Ni and/or Pd, disposed on the coked catalyst can be agglomerated as compared to the calcined/regenerated catalyst prior to contact with the first hydrocarbon-containing feed. It is believed that during combustion of at least a portion of the coke on the coked catalyst that at least a portion of the Pt and, if present, any Ni and/or Pd can be re-dispersed about the support. Re-dispersing at least a portion of any agglomerated Pt and, if present, Ni and/or Pd can increase the activity and improve the stability of the catalyst over many cycles.

In some embodiments, at least a portion of the Pt and, if present, Ni and/or Pd in the regenerated catalyst can be at a higher oxidized state as compared to the Pt and, if present, Ni and/or Pd in the catalyst contacted with the first hydrocarbon-containing feed and as compared to the Pt and, if present, Ni and/or Pd in the coked catalyst. As such, as noted above, in some embodiments the process can optionally include contacting at least a portion of the regenerated catalyst with a reducing gas to produce a regenerated and reduced catalyst. Suitable reducing gases (reducing agent) can be or can include, but are not limited to, H₂, CO, CH₄, C₂H₆, C₃H₈, C₂H₄, C₃H₆, steam, or a mixture thereof. In some embodiments, the reducing agent can be mixed with an inert gas such as Ar, Ne, He, N₂, CO₂, H₂O or a mixture thereof. In such embodiments, at least a portion of the Pt and, if present Ni and/or Pd, in the regenerated and reduced catalyst can be reduced to a lower oxidation state, e.g., the elemental state, as compared to the Pt and, if present, Ni and/or Pd in the regenerated catalyst. In this embodiment, the additional quantity of the hydrocarbon-containing feed can be contacted with at least a portion of the regenerated catalyst and/or at least a portion of the regenerated and reduced catalyst.

In some embodiments, the regenerated catalyst and the reducing gas can be contacted at a temperature in a range from 400° C., 450° C., 500° C., 550° C., 600° C., 620° C., 650° C., or 670° C. to 720° C., 750° C., 800° C., or 900° C. The regenerated catalyst and the reducing gas can be contacted for a time period in a range from 1 second, 5 seconds, 10 seconds, 20 seconds, 30 seconds, or 1 minute to 10 minutes, 30 minutes, or 60 minutes. The regenerated catalyst and reducing gas can be contacted at a reducing agent partial pressure of 20 kPa-absolute, 50 kPa-absolute, or 100 kPa-absolute, 300 kPa-absolute, 500 kPa.-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute. In other embodiments, the reducing agent partial pressure during contact with the regenerated catalyst can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the regenerated catalyst.

At least a portion of the regenerated catalyst, the regenerated and reduced catalyst, new or fresh catalyst, or a mixture thereof can be contacted with an additional quantity of the first hydrocarbon-containing feed within the reaction or conversion zone to produce additional effluent and additional coked catalyst. As noted above, in some embodiments, the cycle time from the contacting the hydrocarbon-containing feed with the calcined/regenerated catalyst to the contacting the additional quantity of the hydrocarbon-containing feed with at least a portion of the regenerated catalyst, and/or the regenerated and reduced catalyst, and optionally with new or fresh catalyst can be ≤5 hours, ≤4 hours, ≤3 hours, ≤2 hours, ≤1 hour, ≤55 minutes, minutes, or ≤45 minutes.

In sonic embodiments, as noted above, one or more additional feeds, e.g., one or more sweep fluids, can be utilized between flows of the first hydrocarbon-containing feed and the oxidant, between the oxidant and the optional reducing gas if used, between the oxidant and the additional first hydrocarbon-containing feed, and/or between the reducing gas and the additional first hydrocarbon-containing feed. The sweep fluid can, among other things, purge or otherwise urge undesired material from the reactor, such as non-combustible particulates including soot. In some embodiments, the additional feed(s) can be inert under the dehydrogenation, dehydroaromatization, and dehydrocyclization, combustion, and/or reducing conditions. Suitable sweep fluids can be or can include, but are not limited to, N₂, He, Ar, CO₂, H₂O, CO₂, CH₄, or a mixture thereof. In some embodiments, if the process utilizes a sweep fluid the duration or time period the sweep fluid is used can be in a range from 1 second, 5 seconds, 10 seconds, 20 seconds, 30 seconds, or 1 minute to 10 minutes, 30 minutes, or 60 minutes.

In some embodiments, the calcined/regenerated catalyst can remain sufficiently active and stable after many cycles, e.g., at least 15, at least 20, at least 30, at least 40, at least 50, at least 60, at least 70, at least 100 cycles, at least 125 cycles, at least 150 cycles, at least 175 cycles, or at least 200 cycles with each cycle time lasting for ≤5 hours, ≤4 hours, ≤3 hours, ≤2 hours, ≤1 hour, ≤50 minutes, ≤45 minutes, ≤30 minutes, ≤15 minutes, ≤10 minutes, ≤5 minutes, ≤1 minute, ≤30 seconds, or ≤10 seconds. In some embodiments, the cycle time can be from 5 seconds, 30 seconds, 1 minute or 5 minutes to 10 minutes, 20 minutes, 30 minutes, 45 minutes, 50 minutes. 70 minutes, 2 hours, 3 hours, 4 hours, or 5 hours. In some embodiments, after the catalyst performance stabilizes (sometimes the first few cycles can have a relatively poor or a relatively good performance, but the performance can eventually stabilize), the process can produce a first upgraded hydrocarbon product yield, e.g., propylene when the hydrocarbon-containing feed includes propane, at an upgraded hydrocarbon selectivity, e.g., propylene, of ≥75%, ≥80%, ≥85%, or ≥90%, or ≥95% when initially contacted with the first hydrocarbon-containing feed, and can have a second upgraded hydrocarbon product yield upon completion of the last cycle (at least 15 cycles total) that can be at least 90%, at least 93%, at least 95%, at least 97%, at least 98%, at least 99%, at least 99.5%, or at least 100% of the first upgraded hydrocarbon product yield at an upgraded hydrocarbon selectivity, e.g., propylene, of ≥75%, ≥80%, ≥85%, or ≥90%, or >95%.

In some embodiments, when the first hydrocarbon-containing feed includes propane and the upgraded hydrocarbon includes propylene, contacting the hydrocarbon-containing feed with the calcined/regenerated catalyst can produce a propylene yield of ≥48%, ≥49%, ≥50%, ≥51%. ≥52%, ≥53%, ≥54%, ≥55%, ≥56%, ≥57%, ≥58%, ≥59%, ≥60%, ≥61%, ≥62%, ≥63%, ≥64%, ≥65%, ≥66%, ≥67%, ≥68%, or ≥69% at a propylene selectivity of ≥75%, ≥80%, ≥85%, ≥90%, ≥93%, or ≥95%. In some embodiments, when the hydrocarbon-containing feed includes propane and the upgraded hydrocarbon includes propylene, contacting the hydrocarbon-containing feed with the calcined/regenerated. catalyst can produce a propylene yield of at least 48%, at least 49%, at least 50%, at least 51%, at least 52%, at least 53%, at least 55%, at least 57%, at least 60%, at least 62%, at least 63%, at least 64%, at least 65%, at least 66%, at least 67%, at least 68%, or at least 69% at a propylene selectivity of at least 75%, at least 80%, at least 85%, at least 90%, or at least 95% for at least 15, at least 20, at least 30, at least 40, at least 50. at least 60, at least 70, at least 100 cycles, at least 125 cycles, at least 150 cycles, at least 175 cycles, or at least 200 cycles. In other embodiments, when the hydrocarbon-containing feed includes at least 70 vol %, at least 75 vol %, at least 80 vol %, at least 85 vol.%, at least 90 vol %, or at least 95 vol % of propane, based on a total volume of the first hydrocarbon-containing feed, is contacted under a propane partial pressure of at least 20 kPa-absolute, a propylene yield of at least 48%, at least 49%, at least 50%, at least 51%, at least 52%, at least 53%, at least 55%, at least 57%, at least 60%, at least 62%, at least 63%, at least 64%, at least 65%, at least 66%, at least 67%, at least 68%, or at least 69% at a propylene selectivity of at least 75%, at least 80%, at least 85%, at least 90%, or at least 95% can be obtained for at least 15, at least 20, at least 30, at least 40, at least 50, at least 60, at least 70, at least 100 cycles, at least 125 cycles, at least 150 cycles, at least 175 cycles, or at least 200 cycles. It is believed that the propylene yield can be further increased to at least 70%, at least 72%, at least 75%, at least 77%, at least 80%, or at least 82% at a propylene selectivity of at least 75%, at least 80%, at least 85%, at least 90%, or at least 95% for at least 15 cycles, at least 20, at least 30, at least 40, at least 50, at least 60, at least 70, at least 100 cycles, at least 125 cycles, at least 150 cycles, at least 175 cycles, or at least 200 cycles by further optimizing the composition of the support and/or adjusting one or more process conditions. In some embodiments, the propylene yield can be obtained when the calcined/regenerated catalyst is contacted with the hydrocarbon-containing feed at a temperature of at least 620° C., at least 630° C., at least 640° C., at least 650° C., at least 655° C., at least 660° C., at least 670° C., at least 680° C., at least 690° C., at least 700° C., or at least 750° C. for at least 15, at least 20, at least 30, at least 40, at least 50, at least 60, at least 70, at least 100 cycles, at least 125 cycles, ai least 150 cycles, at least 175 cycles, or at least 200 cycles.

Systems suitable for carrying out the processes disclosed herein can include systems that are well-known in the art such as the fixed bed reactors disclosed in WO Publication No. WO2017078894; the fluidized riser reactors and/or downer reactors disclosed in U.S. Pat. Nos. 3,888,762; 7,102,050; 7,195,741; 7,122,160; and 8,653,317; and U.S. Patent Application Publication Nos. 2004/0082824; 2008/0194891; and the reverse flow reactors disclosed in U.S. Pat. No. 8,754,276; U.S. Patent Application Publication No. 2015/0065767; and WO Publication No. WO2013169461.

The first hydrocarbon-containing feed can be or can include, but is not limited to, one or more alkane hydrocarbons, e.g., C₂-C₁₆ linear or branched alkanes and/or C₄-C₁₆ cyclic alkanes, and/or one or more alkyl aromatic hydrocarbons, e.g., C₈-C16 alkyl aromatics. In some embodiments, the first hydrocarbon-containing feed can optionally include 0.1 vol % to 50 vol % of steam, based on a total volume of any C₂-C₁₆ alkanes and any C₈-C₁₆ alkyl aromatics in the hydrocarbon-containing feed. In other embodiments, the first hydrocarbon-containing feed can include <0.1 vol % of steam or can be free of steam, based on the total volume of any C₂-C₁₆ alkanes and any C₂-C₁₆ alkyl aromatics in the hydrocarbon-containing feed.

The C₂-C₁₆ alkanes can be or can include, but are not limited to, ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane, 1,3-dimethylcyclohexane, or a mixture thereof. For example, the first hydrocarbon-containing feed can include propane, which can be dehydrogenated to produce propylene, and/or isobutane, which can be dehydrogenated to produce isobutylene. In another example, the first hydrocarbon-containing feed can include liquid petroleum gas (LP gas), which can be in the gaseous phase when contacted with the catalyst. In some embodiments, the first hydrocarbon in the hydrocarbon-containing feed can be composed of substantially a single alkane such as propane. In some embodiments, the hydrocarbon-containing feed can include 50 mol %, ≥75 mol %, ≥95 mol %, ≥98 mol %, or ≥99 mol % of a single C₂-C₁₆ alkane, e.g., propane, based on a total weight of all hydrocarbons in the first hydrocarbon-containing feed. In some embodiments, the first hydrocarbon-containing feed can include at least 50 vol %, at least 55 vol %, at least 60 vol %, at least 65 vol %, at least 70 vol %, at least 75 vol %, at least 80 vol %, at least 85 vol %, at least 90 vol %, at least 95 vol %, at least 97 vol %, or at least 99 vol % of a single C₂-C₁₆ alkane, e.g., propane, based on a total volume of the first hydrocarbon-containing feed.

The C₈-C₁₆ alkyl aromatics can be or can include, but are not limited to, ethylbenzene, propylbenzene, butylbenzene, one or more ethyl toluenes, or a mixture thereof. In some embodiments, the hydrocarbon-containing feed can include 50 mol %, ≥75 mol %, ≥95 mol %, ≥98 mol %, or ≥99 mol % of a single C₈-C₁₆ alkyl aromatic, e.g., ethylbenzene, based on a total weight of all hydrocarbons in the first hydrocarbon-containing feed. In some embodiments, the ethylbenzene can be dehydrogenated to produce styrene. As such, in some embodiments, the first process for upgrading a hydrocarbon disclosed herein can include propane dehydrogenation, butane dehydrogenation, isobutane dehydrogenation, pentane dehydrogenation, pentane dehydrocyclization to cyciopentadiene, naphtha reforming, ethylbenzene dehydrogenation, ethyltoluene dehydrogenation, and the like.

In some embodiments, the first hydrocarbon-containing feed can be diluted, e.g., with one or more diluents such as one or more inert gases. Suitable inert gases can be or can include, but are not limited to, Ar, Ne, He, N₂, CO₂, CH₄, or a mixture thereof. If the hydrocarbon containing-feed includes a diluent, the hydrocarbon-containing feed can include 0.1 vol %, 0.5 vol %, 1 vol %, or 2 vol % to 3 vol %, 8 vol %, 16 vol %, or 32 vol % of the diluent, based on a total volume of any C₂-C₁₆ alkanes and any C₈-C₁₆ alkyl aromatics in the hydrocarbon-containing feed.

In some embodiments, the first hydrocarbon-containing feed can also include H₂. In some embodiments, when the first hydrocarbon-containing feed includes H₂, a molar ratio of the H₂ to a combined amount of any C₂-C₁₆ alkane and any C₈-C₁₆ alkyl aromatic can be in a range from 0.1, 0.3, 0.5, 0.7, or 1 to 2, 3, 4, 5, 6, 7, 8, 9, or 10.

In some embodiments, the first hydrocarbon-containing feed can be substantially free of any steam, e.g., <0.1 vol % of steam, based on a total volume of any C₂-C₁₆ alkanes and any C₈-C₁₆ alkyl aromatics in the hydrocarbon-containing feed. In other embodiments, the first hydrocarbon-containing feed can include steam. For example, the first hydrocarbon-containing feed can include 0.1 vol %, 0.3 vol %, 0.5 vol %, 0.7 vol %, 1 vol %, 3 vol %, or 5 vol % to 10 vol %, 15 vol %, 20 vol %, 25 vol %, 30 vol %, 35 vol %, 40 vol %, 45 vol %, or 50 vol % of steam, based on a total volume of any C₂-C₁₆ alkalies and any C₈-C₁₆ alkyl aromatics in the first hydrocarbon-containing feed, In other embodiments, the first hydrocarbon-containing feed can include ≤50 vol %, ≤45 vol %, ≤40 vol %, ≤35 vol %, ≤30 vol %, ≤25 vol %, ≤20 vol %, or ≤15 vol % of steam, based on a total volume of any C₂-C₁₆ alkanes and any C₈-C16 alkyl aromatics in the first hydrocarbon-containing feed. In other embodiments, the first hydrocarbon-containing feed can include at least 1 vol %, at least 3 vol %, at least 5 vol %, at least 10 vol %, at least 15 vol %, at least 20 vol %, at least 25 vol %, or at least 30 vol % of steam, based on a total volume of any C₂-C₁₆ alkanes and any C₈-C₁₆ alkyl aromatics in the first hydrocarbon-containing feed.

In some embodiments, the first hydrocarbon-containing feed can include sulfur. For example, the first hydrocarbon-containing feed can include sulfur in a range from 0.5 ppm, 1 ppm, 5 ppm, 10 ppm, 20 ppm 30 ppm, 40 ppm, 50 ppm, 60 ppm, 70 ppm, or 80 ppm to 100 ppm, 150 ppm, 200 ppm, 300 ppm, 400 ppm, or 500 ppm. In other embodiments, the first hydrocarbon-containing feed can include sulfur in a range from 1 ppm to 10 ppm, 10 ppm to 20 ppm, 20 ppm to 50 ppm, 50 ppm to 100 ppm, or 100 ppm to 500 ppm. The sulfur, if present in the first hydrocarbon-containing feed, can be or can include, but is not limited to, H2S, dimethyl disulfide, as one or more mercaptans, or any mixture thereof.

In some embodiments, the first hydrocarbon-containing feed can be substantially free or free of molecular oxygen. In some embodiments, the first hydrocarbon-containing feed can include ≤5 mol %, ≤3 mol %, or ≤1 mol % of molecular oxygen (O2). It is believed that providing a first hydrocarbon-containing feed substantially-free of molecular oxygen substantially prevents oxidative reactions that would otherwise consume at least a portion of the alkane and/or the alkyl aromatic in the first hydrocarbon-containing feed.

### Recovery and Use of the First Upgraded Hydrocarbon

In some embodiments, the first upgraded hydrocarbon can include at least one upgraded hydrocarbon, e.g., an olefin, water, unreacted hydrocarbons, molecular hydrogen, etc. The first upgraded hydrocarbon can be recovered or otherwise obtained via any convenient process, e.g., by one or more conventional processes. One such process can include cooling and/or compressing the effluent to condense at least a portion of any water and any heavy hydrocarbon that may be present, leaving the olefin and any unreacted alkane or alkyl aromatic primarily in the vapor phase. Olefin and unreacted alkane or alkyl aromatic hydrocarbons can then be removed from the reaction product in one or more separator drums. For example, one or more splitters or distillation columns can be used to separate the dehydrogenated product from the unreacted first hydrocarbon-containing feed.

In some embodiments, a recovered olefin, e.g., propylene, can be used for producing polymer, e.g., recovered propylene can be polymerized to produce polymer having segments or units derived from the recovered propylene such as polypropylene, ethylene-propylene copolymer, etc. Recovered isobutene can be used, e.g., for producing one or more of: an oxygenate such as methyl tert-butyl ether, fuel additives such as dlisobutene, synthetic elastomeric polymer such as butyl rubber, etc.

### A Second Process for Upgrading a Hydrocarbon

The second process for upgrading a hydrocarbon can include contacting a second hydrocarbon-containing feed with the calcined catalyst that includes the catalyst particles that include Pt and optionally the promoter disposed on the support to effect reforming of at least a portion of the second hydrocarbon-containing feed to produce a coked catalyst and an effluent that can include carbon monoxide and molecular hydrogen. The calcin.ed catalyst and the second hydrocarbon-containing feed can be contacted with one another within any suitable environment such as one or more reaction or conversion zones disposed within one or more reactors to produce the effluent and the coked catalyst. The reaction or conversion zone can be disposed or otherwise located within one or more fixed bed reactors, one or more fluidized or moving bed reactors, one or more reverse flow reactors, or any combination thereof. For clarity and ease of description, the reforming reaction will be discussed in the context of a fluidized bed reactor, but it should be understood that fixed bed reactors, reverse flow or moving bed reactors, or any other reactor can be used to carry out the reforming of the second hydrocarbon-containing feed.

The reforming reaction can be used to produce reformed hydrocarbons via a continuous reaction process or a discontinuous reaction process. In some embodiments, the reaction process can include a reforming step, e.g., an endothermic reaction, and a regeneration step, e.g., an exothermic reaction, that operate continuously while the fluidized catalyst is transported in-between the reforming and regeneration zone of the reactor. The endothermic reaction can include hydrocarbon reforming in the presence of the calcined catalyst. Fresh hydrocarbon and regenerated fluidized catalyst particles can enter the reforming zone. After spending some time in the reforming zone, the hydrocarbon can be at least partially converted to a reforming product that can exit the reforming zone together with the spent catalyst. The reforming product and unreacted feed can be separated from the spent catalyst by one or more separating devices. While the reforming product and unreacted feed from the separating devices go downstream for further purification, the spent catalyst can be sent to the regeneration zone for regeneration. The exothermic regeneration reaction can be the reaction of an oxidant and, optionally a fuel, under combustion conditions to produce a regenerated catalyst and a flue gas. After regeneration, the regenerated catalyst can be separated from the flue gas by one or more separating devices and can be transported hack to the reforming zone, joining more hydrocarbon feed to enter the reforming zone to initiate more reforming reaction. The reforming step can convert CO₂ and/or H₂ O and hydrocarbons, e.g., CH₄, to a synthesis gas that includes H₂ and CO. The regeneration step can combust reactants, e.g., coke disposed on the spent catalyst and/or the optional fuel and an oxidant, to generate heat that heats up the regenerated catalyst that can provide heat that can be used to drive the reforming reaction. In some embodiments, the catalyst can be heated to an average temperature in a range of from 600° C., 700° C., or 800° C. to 1,000° C., 1,300° C., or 1,600° C. during the regeneration step.

Illustrative fuels can be or can include, but are not limited to, hydrocarbons, e.g., methane, ethane, propane, butane, pentane, or hydrocarbon containing streams, e.g., natural gas, molecular hydrogen, fuel oil, heavy fuel oil, gasoline, diesel, kerosene, distillate, andlor other combustible compounds. The oxidant can be or can include O₂. In some embodiments, the oxidant can be or can include air, O₂ enriched air, O₂ depleted air, or any other suitable O₂ containing stream.

The regeneration of the catalyst can correspond to removal of coke from the catalyst particles. In some embodiments, during reforming, a portion of the feed introduced into the reforming zone can form coke. This coke can potentially block access to the catalytic sites (such as metal sites) of the catalyst. During regeneration at least a portion of the coke generated during reforming can be removed as CO or CO₂. The regeneration of the catalyst can also correspond to re-dispersion of any agglomerated active phase of the catalyst such as Pt.

The second hydrocarbon-containing feed can be or can include, but is not limited to, one or more reformable C₁-C₁₆ hydrocarbons such as alkanes, alkenes, cycloalkanes, alkylaromatics, or any mixture thereof In some embodiments, the second hydrocarbon-containinu stream can be or can include methane, ethane, propane, butane, pentane, or a mixture thereof. In some embodiments, the second hydrocarbon-containing feed can be exposed to the catalyst under a pressure of less than 35 kPag. For example, the second hydrocarbon-containing feed can be exposed to the catalyst under a pressure in a range of from kPag, 2 kPag, 3.5 kPag, 5 kPag, or 10 kPag to 15 kPag, 20 kPag, 25 kPag, or 30 kPag. In other embodiments, the second hydrocarbon-containing feed can be exposed to the catalyst under a pressure in a range of from 35 kPag to 15 MPag. In still other embodiments, the second hydrocarbon-containing feed can be exposed to the catalyst under a pressure in a range of from kPag, 2 kPag, 5 kPag, 20 kPag, 35 kPag, 50 kPag, or 100 kPag to 200 kPag, 1 MPag, 3 MPag, 5 MPag, 10 MPag, or 15 MPag. In still other embodiments, the second hydrocarbon-containing feed can be exposed to the catalyst under a pressure of less than 2.8 MPag, less than 2.5 MPag, less than 2.2 MPag, or less than 2 MPag.

The reforming reaction of the second hydrocarbon-containing feed, e.g., CH₄, can occur in the presence of H₂ O (steam-reforming), in the presence of CO₂ (dry-reforming), or in the presence of both H₂ O and CO₂ (bi-reforming). Examples of stoichiometry for steam, dry, and bi-reforming of CH₄ are shown in equations (1)-(3).

(1) Dry-Reforming:

C₄+CO₂=2CO+2H₂

(2) Steam-Reforming:

CH₄+H₂O═CO+3H₂

(3) Bi-Reforming:

3CH₄+2H₂O+CO₂4CO+8H₂

As shown in equations (1)-(3), dry reforming can produce lower ratios of H₂ to CO than steam reforming. Reforming reactions performed with only steam can generally produce a synthesis gas having a H₂:CO molar ratio of around 3, such as 2.5 to 3.5. In contrast, reforming reactions performed with only CO₂ can generally produce a synthesis gas having a H₂:CO molar ratio of roughly 1 or even lower. By using a combination of CO₂ and H₂O during reforming, the reforming reaction can be controlled to generate a wide variety of H₂ to CO ratios in a resulting synthesis gas.

It should be noted that the ratio of H2 to CO in a synthesis gas can also he dependent on the water gas shift equilibrium. Although the stoichiometry in Equations (1)-(3) shows ratios of roughly 1 or roughly 3 for dry reforming and steam reforming, respectively, the equilibrium amounts of H₂ and CO in a synthesis gas can be different from the reaction stoichiometry. The equilibrium amounts can be determined based on the water gas shift equilibrium, which relates the concentrations of H₂, CO, CO₂ and H₂O based on the reaction shown in equation (4).

(4) H₂O+CO⇄H₂+CO₂

In some embodiments, the calcined catalyst can also serve as water vias shift catalysts. Thus, if a reaction environment for producing H₂ and CO also includes H₂O and/or CO₂, the initial stoichiometry from the reforming reaction may be altered based on the water gas shift equilibrium. However, this equilibrium is also temperature dependent, with higher temperatures favoring production of CO and H₂O. As a result, the ratio of H₂ to CO that is generated when forming synthesis gas is constrained by the water gas shift equilibrium at the temperature in the reaction zone when the synthesis gas is produced.

The ability to adjust the H₂:CO molar ratio of the synthesis gas provides a flexible process that can be combined with a wide variety of synthesis gas upgrading processes. Illustrative synthesis gas upgrading processes can include, but are not limited to, Fischer-Tropsch processes, methanol and/or other alcohol synthesis, e.g., one or more C₁-C₄ alcohols, fermentation processes, separation processes that can separate hydrogen to produce a H₂-rich product, dimethyl ether, and combinations thereof, These synthesis gas upgrading processes are well-known to persons having ordinary skill in the art. In some embodiments, the upgraded product can include, but is not limited to, methanol, syncrude, diesel, lubricants, waxes, olefins, dimethyl ether, other chemicals, or any combination thereof.

Systems suitable for carrying out the reforming of the second hydrocarbon-containing feed can include systems that are well-known in the art such as the fixed bed reactors disclosed in WO Publication No. WO2017078894; the fluidized riser reactors and/or downer reactors disclosed in U.S. Pat. Nos. 3,888,762; 7,102,050; 7,195,741; 7,122,160; and 8,653,317; and U.S. Patent Application Publication Nos. 2004/0082824; 2008/0194891; and the reverse flow reactors disclosed in U.S. Pat. Nos.: 7,740,829; 8,551,444; 8,754,276; 9,687,803; and and U.S. Patent Application Publication Nos.: 2015/0065767 and 2017/0137285; and WO Publication No. WO2013169461.

### EXAMPLES

The foregoing discussion can be further described with reference to the following non-limiting examples.

Synthesized Catalyst 1 was prepared according to the following procedure. Calcined hydrotalcite support particles (23.0 g; MgO:Al₂O₃=71/29 w/w) that had physical properties consistent with those of Geldart A fluidizable particles were mixed with 40 ml of deionized (DI) water to make a slurry. An aqueous mixture that contained 0.38 g of an 8% chroloplatinic acid solution; 2.97 g of 23.65% tin(IV) chloride pentahydrate, and 20 ml of DI water was prepared. Under stirring, the aqueous mixture was added slowly to the slurry. After finishing addition, the mixture was stirred for an additional 10 minutes before the solid fraction was recovered by filtration. The solids were then dried in air at 110° C. for 6 hours. After drying, the solids still contained a significant amount of volatile compounds andlor compounds that can form volatile compounds if subjected to thermal treatments at temperatures higher than 110° C. The non-volatile weight of the catalyst was quantified by thermogravimetric analysis (TGA) in an oxidative environment (air) by heating the synthesized catalyst to a temperature of 900° C. Synthesized Catalyst 1 had a Pt and Sn loading of approximately 0.05 wt % and 1.0 wt %, respectively, based on the non-volatile weight of the catalyst.

Nine separate samples of Synthesized Catalyst 1 were obtained and separately calcined under nine different calcination processes to obtain calcined catalysts (Ex. 1-9). The calcination processes were as follows.

Calcination 1 (Ex. 1; (O)): 1. Under a flow of 46.6 sccm of air, the reaction zone temperature was increased from room temperature to 800° C. at 5° C./min and the catalyst particles were calcined at 800° C. for 12 hours to produce the calcined catalyst particles.

Calcination 2 (Ex. 2; (O)): 1. Under a flow of 46.6 sccm of air, the reaction zone temperature was increased from room temperature to 550° C. at 30° C./min and the catalyst particles were calcined at 550° C. for 0.5 hours to produce the calcined catalyst panicles.

Calcination 3 (Ex. 3; (OR)): 1. Under a flow of 46.6 sccm of air, the reaction zone temperature was increased from room temperature to 110° C. at 30° C./min and the catalyst particles were dried at 110° C. for 0.5 hours. 2. The reaction zone temperature was then increased from 110° C. to 600° C. at 30° C./min under a flow of inert gas. 3. Under a flow of 46.6 sccm of 10% H₂ in argon, the catalyst particles were calcined at 600° C. for 2.5 hours to produce the calcined catalyst particles.

Calcination 4 (Ex. 4; (OR)): 1. Under a flow of 46.6 sccm of air, the reaction zone temperature was increased from room temperature to 450° C. at 30° C./min and the catalyst particles were calcined at 450° C. for 0.5 hours. 2. The reaction zone temperature was increased from 450° C. to 600° C. at 30° C./min under a flow of inert gas. 3. Under a flow of 46.6 sccm of 10% H₂ in argon, the catalyst particles were calcined at 600° C. for 2.5 hours to produce the calcined catalyst particles.

Calcination 5 (Ex. 5; (OR)): 1. Under a flow of 46.6 sccm of air, the reaction zone temperature was increased from room temperature to 550° C. at 30° C./min and the catalyst particles were calcined at 550° C. for 0.5 h. 2. The reaction zone temperature was then increased from 550° C. to 600° C. at 30° C./min under a flow of inert. 3. Under a flow of 46.6 sccm of 10% H₂ in argon, the catalyst particles were calcined at 600° C. for 2.5 hours to produce the calcined catalyst particles.

Calcination 6 (Ex. 6; (OR)): 1. Under a flow of 46.6 sccm of air, the reaction zone temperature was increased from room temperature to 800° C. at 30° C./min and the catalyst particles were calcined at 800° C. for 0.5 hours. 2. The reaction zone temperature was then decreased from 800° C. to 600° C. at 30° C./min under a flow of inert gas. 3. Under a flow of 46.6 sccm of 10% H₂ in argon, the catalyst particles were calcined at 600° C. for 2.5 hours to produce the calcined catalyst particles.

Calcination 7 (Ex. 7; (OR)): 1. Under a flow of 46.6 sccm of air, the reaction zone temperature was increased from room temperature to 550° C. at 30° C./min and the catalyst particles were calcined at 550° C. for 0.5 hours. 2. The reaction zone temperature was then increased from 550° C. to 600° C. at 30° C./min under a flow of inert gas. 3, Under a flow of 46.6 sccm of 10% H₂ in argon, the catalyst particles were calcined at 600° C. for 1.25 hours to produce the calcined catalyst particles.

Calcination 8 (Ex. 8; (OR)): 1. Under a flow of 46.6 scent of air, the reaction zone temperature was increased from room temperature to 550° C. at 30° C./min and the catalyst particles were calcined at 550° C. for 0.5 hours. 2. The reaction zone temperature was then increased from 550° C. to 600° C. at 30° C./min under a flow of inert gas. 3. Under allow of 46.6 sccm of 10% H₂ in argon, the catalyst particles were calcined at 600° C. for 5 hours to produce the calcined catalyst particles.

Calcination 9 (Ex. 9; (OROR)): 1. Under a flow of 46.6 sccm of air, the reaction zone temperature was increased from room temperature to 550° C. at 30° C./min and the catalyst particles were calcined at 550° C. for 0.25 hours. 2. The reaction zone temperature was then increased from 550° C. to 600° C. at 30° C./min under a flow of inert gas. 3. Under a flow of 46.6 sccm of 10% H₂ in argon, the catalyst particles were calcined at 600° C. for 0.625 hours. 4. The system was then purged with an inert gas. 5. The reaction zone temperature was then decreased from 600° C. to 550° C. at 30° C./min under a flow of 46.6 sccm of air and the catalyst particles were calcined at 550° C. for 0.25 hours. 6. The reaction zone temperature was increased from 550° C. to 600° C. at 30° C.; ruin under a flow of inert gas. 7. Under a flow of 46.6 sccm of 10% H₂ in argon, the catalyst particles were calcined at 600° C. for 0.625 hours to produce the calcined catalyst particles.

Fixed bed experiments were conducted at approximately 100 kPa-absolute that used the calcined catalysts of Exs. 1-9. A gas chromatograph (GC) was used to measure the composition of the reactor effluents. The concentrations of each component in the reactor effluents were then used to calculate the C₃H₆ yield and selectivity. The C₃H₆ yield and the selectivity at the beginning of the reaction is denoted as Yᵢₙᵢ and Sᵢₙᵢ, respectively, and reported as percentages in the tables below. The C₃H₆ yield and selectivity, as reported in the examples, were calculated on the carbon mole basis.

In each example, 0.3 g of catalyst (on a non-volatile basis) was mixed with an appropriate amount of silicon carbide and loaded into a quartz reactor. The amount of SiC was determined so that the catalyst bed (catalyst+SiC) overlapped with the isothermal zone of the quartz reactor and the catalyst bed was largely isothermal during operation. The dead volume of the reactor was filled with quartz rods.

The process steps for Examples were as follows: 1. The system was flushed with an inert gas. 2. 83.9 sccm of dry air was passed through a by-pass of the reaction zone, while an inert gas was passed through the reaction zone, 3. The reaction zone was heated to a regeneration temperature of 800° C. 4. 83.9 sccm of air was then passed through the reaction zone for 10 min to regenerate the catalyst 5. The system was flushed with an inert gas. 6. 46.6 sccm of a H₂ containing gas (10 vol % H₂ and 90 vol % Ar) was passed through the by-pass of the reaction zone for a certain period of time, while an inert gas was passed through the reaction zone. This was then followed by flowing the H₂ containing gas through the reaction zone at 800° C. for 3 s. The system was flushed with an inert gas. During this process, the temperature of the reaction zone was changed from 800° C. to a reaction temperature of 670° C. 7. A hydrocarbon-containing (HCgas) feed that included 81 vol % of C₃H₈, 9 vol % of Ar and 10 vol % of steam at a flow rate of 17.6 sccm was passed through the by-pass of the reaction zone for a certain period of time, while an inert gas was passed through the reaction zone. The hydrocarbon-containing feed was then passed through the reaction zone at 670° C. for 10 min. GC sampling of the reaction effluent started as soon as the feed was switched from the by-pass of the reaction zone to the reaction zone, 8. The above process steps 1-7 were repeated for 14 cycles. Stable performance was obtained after 8 cycles.

TABLE 1
Catalyst 1

| | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Calcination | (O) | (O) | (R) | (OR) | (OR) | (OR) | (OR) | (OR) | (OROR) |
| Cycle 1, Yᵢₙᵢ | 7.9 | 5.5 | 45.7 | 52.0 | 51.4 | 35.3 | 52.1 | 52.0 | 52.3 |
| Cycle 1, Sᵢₙᵢ | 75.3 | 72.6 | 95.5 | 96.6 | 96.0 | 93.4 | 96.3 | 95.5 | 95.4 |
| Cycle 2, Yᵢₙᵢ | 36.1 | 54.9 | 61.0 | 63.8 | 65.6 | 53.8 | 63.7 | 65.1 | 66.5 |
| Cycle 2, Sᵢₙᵢ | 90.8 | 94.1 | 94.8 | 95.9 | 94.3 | 93.8 | 95.5 | 95.0 | 94.8 |
| Cycle 14, Yᵢₙᵢ | 60.6 | 62.7 | 64.0 | 66.2 | 67.9 | 59.4 | 66.5 | 67.5 | 69.1 |
| Cycle 14, Sᵢₙᵢ | 92.1 | 93.5 | 93.1 | 94.1 | 93.2 | 93.5 | 94.1 | 93.2 | 92.7 |

Comparison between Calcinations 1 and 2 vs. 3-9 suggest that a reductive calcination can be more effective than oxidative calcination. Comparison between Calcinations 1 and 2 vs. 4-9 shows that a reductive calcination following an oxidative calcination may help to significantly increase the C₃H₆ yield, For example, Calcinations 4, 5, 7, and 8 resulted in a better performing catalyst. while Calcination 6 led to a slightly more poorly performing catalyst. Comparison between Calcinations 4-8 shows that the oxidative calcination should preferably not be carried out at a temperature that is too high or too low. Comparison between Calcinations 5, 7, and 8 shows that the reductive calcination should preferably be neither too long nor too short. Comparison between Calcinations 5 and 9 shows that there can be advantages gained by breaking the oxidative and reductive calcinations into two repeated cycles, while keeping the total duration of time constant.

Three additional samples of Synthesized Catalyst I were obtained and calcined under three additional Calcination processes to obtain calcined catalysts (Ex. 10-12). The Calcination processes were as follows.

Calcination 10 (Ex. 10, (OROR)); 1. Under a flow of 46.6 sccm of air, the reaction zone temperature was increased from room temperature to 600° C. at 30° C./min and the catalyst particles were calcined at 600° C. for 0.25 hours. 2. The system was then purged with an inert gas. 3. Under a flow of 46.6 sccm of 10% H₂ in argon, the catalyst particles were calcined at 600° C. for 0.625 hours. 4. The system was purged with an inert gas. 5. Under a flow of 46.6 sccm of air the catalyst particles were calcined at 600° C. for 0.25 hours. 6. The system was then purged with an inert gas. 7. Under a flow of 46.6 sccm of 10% H₂ in argon, the catalyst particles were calcined at 600° C. for 0.625 hours to produce the calcined catalyst particles,

Calcination 11 (Ex. 11; (OROR)): 1. Under a flow of 46.6 sccm of air, the reaction zone temperature was increased from room temperature to 550° C. at 30° C./min and the catalyst particles were calcined at 550° C. for 0.25 hours. 2. The reaction zone temperature was increased from 550° C. to 650° C. at 30° C./min under a flow of inert. 3. Under a flow of 46.6 sccm of 10% H₂ in argon, the catalyst particles were calcined at 650° C. for 0.625 hours. 4. The system was purged with an inert gas. 5. The reaction zone temperature was decreased from 650° C. to 550° C. at 30° C./min under a flow of 46.6 sccm of air and the catalyst particles were calcined at 550° C. for 0.25 hours. 6. The reaction zone temperature was increased from 550° C. to 650° C. at 30° C./min under a flow of inert gas. 7. Under a flow of 46.6 sccm of 10% H₂ in argon, the catalyst particles were calcined at 650° C. for 0.625 hours to produce the calcined catalyst particles.

Calcination 12 (Ex. 12; (OROROROR)): 1. Under a flow of 46.6 sccm of air, the reaction zone temperature was increased from room temperature to 600° C. at 30° C./min and the catalyst particles were calcined at 600° C., for 5 minutes. 2. The system was purged with an inert gas. 3. Under a flow of 46.6 sccm of 10% H₂ in argon, the catalyst particles were calcined at 600° C. for 15 minutes. 4. The system, was purged with an inert gas. 5. Under a flow of 46.6 sccm of air the catalyst particles were calcined. at 600° C. for 5 minutes. 6. The system was purged with an inert gas. 7. Under a flow of 46.6 sccm of 10% H₂ in argon, the catalyst particles were calcined at 600° C. for 15 minutes. 8. Steps 4-7 were repeated two (2) times. 9. The system was purged with an inert gas. 10. Under a flow of 46.6 sccm of air, the catalyst particles were calcined at 600° C. for 5 min. 11. The system was purged with an inert gas. 12. Under a flow of 46.6 sccm of 10% H₂ in argon, the catalyst particles were calcined at 600° C. for 20 minutes to produce the calcined catalyst particles.

Fixed bed experiments were conducted at approximately 100 kPa-absolute that used the calcined catalysts of Exs. 10-12. The same procedure used for the calcined catalysts of Exs. 1-9 was used for the calcined catalysts of Exs, 10-12. The results are shown in Table 2 below, It is noted that the reactor used to carry out the fixed bed experiments in Exs. 10-12 was a different reactor than was used in Exs. 1-9. As such, the results shown in Table 1 and Table 2 should not be compared to one another in terms of the performance of the catalyst because the reactors were not the same.

TABLE 2

| | Ex. 10 | Ex. 11 | Ex. 12 |
|---|---|---|---|
| Calcination | (OROR) | (OROR) | (OROROROROR) |
| Cycle 1, Yᵢₙᵢ | 50.7 | 51.9 | 48.9 |
| Cycle 1, Sᵢₙᵢ | 95.7 | 95.4 | 88.7 |
| Cycle 2, Yᵢₙᵢ | 64.5 | 65.3 | 61.6 |
| Cycle 2, Sᵢₙᵢ | 95.0 | 94.8 | 93.6 |
| Cycle 14, Yᵢₙᵢ | 67.7 | 68.6 | 64.9 |
| Cycle 14, Sᵢₙᵢ | 93.3 | 93.3 | 92.9 |

Comparison between Exs, 10 and 12 shows that breaking the oxidative and reductive calcination into 5 repeated cycles, while keeping the total duration constant, did not provide an advantage in terms of C₃H₆ yield. Comparison between Ex. 10 and 11 shows that reducing the temperature during the oxidative calcination from 600° C. to 550° C. and increasing the temperature during the reductive calcination from 600° C. to 650° C. increased the C₃H₆ yield.

Synthesized Catalyst 2 was prepared according to the following procedure. Calcined hydrotalcite support particles (46.0 g; MgO: Al₂O₃=77/23 w/w) that had physical properties consistent with those of Geldart A fluidizable particles was mixed with 80 ml of DI water to make a slurry. An aqueous mixture that contained 0.32 g of an 8% chroloplatinic acid solution, 5.95 g of 23.65% tin(IV) chloride pentahydrate, and 40 ml of DI water was prepared. Under stirring, the aqueous mixture was added slowly to the slurry. After finishing addition, the mixture was stirred for an additional 10 minutes before the solid fraction was recovered by filtration. The recovered solids was equilibrated at room temperature for 30 minutes and then dried in air at 300° C. for 0.5 hours. After drying, the solid still contained a significant amount of volatile compounds or compounds that can form volatile compounds if subjected to thermal treatments at temperatures higher than 300° C. The non-volatile weight of the catalyst was quantified by thermogravimetric analysis (TGA) in an oxidative environment (air) by heating the synthesized catalyst to a temperature of 900° C. Synthesized Catalyst 2 had a Pt and Sn loading of approximately 0.025 wt % and 1.0 wt %, respectively, based on the non-volatile weight of the catalyst.

Four separate samples of Synthesized Catalyst 2 were obtained and separately calcined under four different calcination processes to obtain calcined catalysts (Ex. 13-16). The calcination processes were as follows,

Calcination 13 (Ex. 13; (O)): 1. Under a flow of 46.6 sccm of air, the reaction zone temperature was increased from room temperature to 550° C. at 30° C./min and the catalyst particles were calcined at 550° C. for 0.5 hours to produce the calcined catalyst particles.

Calcination 14 (Ex. 14; (OR)): 1. Under a flow of 46.6 sccm of air, the reaction zone temperature was increased from room temperature to 550° C. at 30° C./min and the catalyst particles were calcined at 550° C. for 0.5 hours. 2. The reaction zone temperature was then increased from 550° C., to 650° C. at 30° C./min under a flow of inert gas. 3, Under a flow of 46.6 sccm of 100% H₂, the catalyst particles were calcined at 650° C. for 1.25 hours to produce the calcined catalyst particles.

Calcination 15 (Ex. 16; (OR)): 1, Under a flow of 46.6 sccm of air, the reaction zone temperature was increased from room temperature to 550° C. at 30° C./min and the catalyst particles were calcined at 550° C. for 0.5 hours. 2. The reaction zone temperature was then increased from 550° C. to 650° C. at 30° C./min under a flow of inert gas. 3. Under a flow of 46.6 sccm of 10% H₂ in argon, the catalyst particles were calcined at 650° C. for 1.25 hours to produce the calcined catalyst particles.

Calcination 16 (Ex. 16; (ORO)): 1. Under a flow of 46.6 sccm of air, the reaction zone temperature was increased from room temperature to 550° C. at 30° C./min and the catalyst particles were calcined at 550° C. for 0.5 hours. 2. The reaction zone temperature was increased from 550° C. to 650° C. at 30° C./min under a flow of inert gas. 3. Under a flow of 46.6 sccm of 100% H₂, the catalyst particles were calcined at 650° C. for 1.25 hours. 4. The system was purged with an inert gas. 5. The reaction zone temperature was decreased from 650° C. to 550° C. at 30° C./min under a. flow of 46.6 sccm of air and the catalyst particles were calcined at 550° C. for 0.5 hours to produce the calcined catalyst particles.

Fixed bed experiments were conducted at approximately 100 kPa-absolute that used the calcined catalysts of Exs. 13-16. The same procedure used for the calcined catalysts of Exs. 1-9 was used for the calcined catalysts of Exs. 13-16.

TABLE 3

| | Ex. 13 | Ex. 14 | Ex. 15 | Ex. 16 |
|---|---|---|---|---|
| Calcination | (O) | (OR) | (OR) | (ORO) |
| Reducing Gas | n/a | 100% H₂ | 10% H₂ | 100% H₂ |
| Cycle 1, Yᵢₙᵢ | 5.9 | 42.9 | 47.8 | 39.4 |
| Cycle 1, Sᵢₙᵢ | 73.4 | 96.2 | 95.6 | 95.7 |
| Cycle 2, Yᵢₙᵢ | 46.1 | 61.3 | 59.4 | 58.5 |
| Cycle 2, Sᵢₙᵢ | 94.7 | 95.9 | 95.1 | 95.9 |
| Cycle 14, Yᵢₙᵢ | 61.3 | 68.8 | 60.0 | 68.3 |
| Cycle 14, Sᵢₙᵢ | 94.6 | 94.2 | 93.7 | 94.8 |

Table 3 suggests that a. combination of reductive/oxidative calcination can also increase the C₃H₆ yield for a catalyst with 0.025 wt % of Pt and a slightly different MgO:Al₂O₃ ratio as compared to the Synthesized Catalyst 1. However, compared with Catalyst 1, 100% H₂, instead of 10% H₂ was needed for Synthesized Catalyst 2 to achieve the highest C₃H₆ yield, presumably due to the higher MgO:Al₂O₃ ratio of Catalyst 2 (77/23 w/w) vs. that of Sytnesized Catalyst 1 (71/29 w/w).

### Listing of Embodiments

This disclosure may further include the following non-limiting embodiments.

A1. A process for calcining a catalyst, comprising: subjecting a synthesized catalyst comprising Pt disposed on a support to a calcination process comprising heating the synthesized catalyst ander a. first atmosphere at a first temperature for a first time period and heating the synthesized catalyst under a second atmosphere at a second temperature for a second time period to produce a calcined catalyst, wherein: the synthesized catalyst comprises <0.05 wt % of the Pt, based on the non-volatile weight of the catalyst, and (i) the first atmosphere comprises a first oxidizing gas, the first temperature is in a range from 350° C. to 850° C., and the first time period is in a range from 30 seconds to 10 hours and the second atmosphere comprises a first reducing gas, the second temperature is in a range from 500° C. to 850° C., and the second time period is in a range from 30 seconds to 10 hours, or (ii) the first atmosphere comprises a first reducing gas, the first temperature is in a range from 500° C. to 850° C., and the first time period is in a range from 30 seconds to 10 hours and the second atmosphere comprises a first oxidizing gas, the second temperature is in a range from 350° C. to 850° C., and the second time period is in a range from 30 seconds to 10 hours.

A2. The process of A1, wherein the first oxidizing gas comprises, O₂, O₃, CO₂, steam, or a mixture thereof, and wherein the first reducing gas comprises H₂, CO, CH₄, C₂H₆, C₃H₈, C₂H₄, C₃H₆, steam, or a mixture thereof.

A3. The process of A1 or A2. wherein, when the synthesized catalyst is initially , subjected to the calcination process, the synthesized catalyst comprises one or more volatile compounds, and wherein the one or more volatile compounds comprise adsorbed CO₂, adsorbed H₂O, adsorbed ethanol, or a mixture thereof.

A4. The process of any one of A1 to A3, wherein the first atmosphere comprises the first oxidizing gas and the second atmosphere comprises the first reducing gas, the process further comprising: heating the synthesized catalyst under a third atmosphere at a third temperature for a third time period to produce the calcined catalyst, wherein the third atmosphere comprises a second oxidizing gas, the third temperature is in a range from 350° C. to 850° C., and the third time period is in a range from 30 seconds to 10 hours.

A5. The process of A4, further comprising heating the synthesized catalyst under a fourth atmosphere at a fourth temperature for a fourth period of time to produce the calcined catalyst, wherein the fourth atmosphere comprises a second reducing gas, the fourth temperature is in a range from 500° C. to 850° C., and the fourth time period is in a range from seconds to 10 hours.

A6, The process of A5, wherein the second oxidizing gas comprises, O₂, O₃, CO₂, steam, or a mixture thereof, and wherein the second reducing gas comprises H₂. CO, CH₄, C₂H₆, C₃H₈, C₂H₄, C₃H₆, steam, or a mixture thereof.

A7. The process of any one of A1 to A3, wherein the first atmosphere comprises the first reducing gas and the second atmosphere comprises the first oxidizing gas, the process further comprising: heating the synthesized catalyst under a third atmosphere at a third temperature for a third time period to produce the calcined catalyst, wherein the third atmosphere comprises a second reducing gas, the third temperature is in a range from 500° C. to 850° C., and the third time period is in a range from 30 seconds to 10 hours.

A8. The process of A7, further comprising heating the synthesized catalyst under a fourth atmosphere at a fourth temperature for a fourth period of time to produce the calcined catalyst, wherein the fourth atmosphere comprises a second oxidizing gas, the fourth temperature is in a range from 350° C. to 850° C. and the fourth time period is in a range from 30 seconds to 10 hours.

A9, The process of A8, wherein the second reducing gas comprises H₂, CO, CH₄, C₂H₅, C₃H₈, C₂H₄, C₃H₆, steam, or a mixture thereof, and wherein the second oxidizing gas comprises, O₂, O₃, CO₂, steam, or a mixture thereof.

A10. The process of any one of A1 to A9, wherein: the synthesized catalyst further comprises up to 10 wt % of a promoter comprising Sn, Cu, Au, Ag, Ga, a combination thereof, or a mixture thereof disposed on the support, the support comprises at least 0.5 wt % of a Group 2 element, and all weight percent values are based on the non-volatile weight of the catalyst.

A11. The process of A10, wherein: the Group 2 element comprises Mg, and at least a portion of the Group 2 element is in the form of MgO or a mixed metal oxide comprising Mg.

A12. The process of A10, wherein: the support further comprises a Group 13 element, the promoter comprises Sn, the Group 2 element comprises Mg, the Group 13 element comprises A1, and the support comprises a mixed Mg/Al metal oxide.

A13. The process of any one of A1 to A12, wherein the synthesized catalyst is in the form of particles that have a size and particle density that is consistent with a Geldart A definition of a fluidizable

A14. The process of any one of A1 to A13, wherein the calcined catalyst, when contacted with propane under dehydrogenation conditions, generates a propylene yield of ≥48% at a propylene selectivity of ≥90%.

A15. The process of any one of A1 to A14, wherein a composition of the first atmosphere and a composition of second atmosphere independently remains constant or varies during the first time period and the second time period, respectively.

B1. A process for calcining a catalyst, comprising: subjecting synthesized catalyst particles comprising Pt disposed on a support to a calcination process comprising heating the synthesized catalyst particles under a first atmosphere at a first temperature for a first time period and heating the synthesized catalyst particles under a second atmosphere at a second temperature for a second time period to produce calcined catalyst particles, wherein: the synthesized catalyst particles have a size and particle density that is consistent with a Geldart A definition of a fluidizable solid, and (i) the first atmosphere comprises a first oxidizing gas, the first temperature is in a range from 350° C. to 850° C., and the first time period is in a range from 30 seconds to 10 hours and the second atmosphere comprises a first reducing gas, the second temperature is in a range from 500° C. to 850° C., and the second time period is in a range from 30 seconds to 10 hours, or (ii) the first atmosphere comprises a first reducing vas, the first temperature is in a range from 500° C. to 850° C., and the first time period is in a range from 30 seconds to 10 hours and the second atmosphere comprises a first oxidizing gas, the second temperature is in a range from 350° C. to 850° C., and the second time period is in a range from 30 seconds to 10 hours.

B2. The process of B 1, wherein the first oxidizing gas comprises, O₂, O₃, CO₂, steam, or a mixture thereof, and wherein the first reducing gas comprises H₂, CO, CH₄, C₂H₆, C₃H₈, C₂H₄, C₃H₆, steam, or a mixture thereof.

B3. The process of B1 or B2, wherein, when the catalyst particles are initially subjected to the calcination process, the catalyst particles comprise one or more volatile compounds, and wherein the one or more volatile compounds comprise adsorbed CO₂, adsorbed H2O, adsorbed ethanol, or a mixture thereof.

B4. The process of any one of B1 to B3, wherein the first atmosphere comprises the first oxidizing gas and the second atmosphere comprises the first reducing gas, the process further comprising: heating the synthesized catalyst particles under a third atmosphere at a third temperature for a third time period to produce the calcined catalyst particles, wherein the third atmosphere comprises a second oxidizing gas, the third temperature is in a range from 350° C. to 850° C., and the third time period is in a range from 30 seconds to 10 hours.

B5. The process of claim B4, fitrther comprising heating the synthesized catalyst particles under a fourth atmosphere at a fourth temperature for a fourth period of time to produce the calcined. catalyst particles, wherein the fourth atmosphere comprises a second reducing gas, the fourth temperature is in a range from 500° C. to 850° C., and the fourth time period is in a range from 30 seconds to 10 hours.

B6. The process of B5, wherein the second oxidizing gas comprises, O₂, O₃, CO₂, steam, or a mixture thereof, and wherein the second reducing gas comprises H₂, CO, CH₄, C₂H₆, C₃H₈, C₂H₄, C₃H₆, steam, or a mixture thereof.

B7. The process of any one of B4 to B6, wherein the first atmosphere comprises the first reducing gas and the second atmosphere comprises the first oxidizing gas, the process further comprising: heating the synthesized catalyst particles under a third atmosphere at a third temperature for a third time period to produce the calcined catalyst particles, wherein the third atmosphere comprises a second reducing gas, the third temperature is in a range from 500° C. to 850° C., and the third time period is in a range from 30 seconds to 10 hours.

B8. The process of 137, further comprising heating the synthesized catalyst particles under a fourth atmosphere at a fourth temperature for a fourth period of time to produce the calcined catalyst particles, wherein the fourth atmosphere comprises a second oxidizing gas, the fourth temperature is in a range from 350° C. to 850° C., and the fourth time period is in a range from 30 seconds to 10 hours.

B9. The process of B8, wherein the second reducing gas comprises H₂, CO, CH₄, C₂H₆, C₃H₈, C₂H₄, C₃H₆, steam, or a mixture thereof, and wherein the second oxidizing gas comprises, O₂, O₃, CO₂, steam, or a mixture thereof.

B10, The process of any one of B1 to B9, wherein: the synthesized catalyst particles further comprise up to 10 wt % of a promoter comprising Sn, Cu, Au, Ag, Ga, a combination thereof, or a mixture thereof disposed on the support, the support comprises at least 0.5 wt % of a Group 2 element, and all weight percent values are based on the non-volatile weight of the catalyst.

B11. The process of B10, wherein: the Group 2 element comprises Mg, and at least a portion of the Group 2 element is in the form of MgO or a mixed metal oxide comprising Mg.

B12. The process of B10, wherein: the support further comprises a Group 13 element, the promoter comprises Sn, the Group 2 element comprises Mg, the Group 13 element comprises A1, and the support comprises a mixed Mg/Al metal oxide.

B13. The process of any one of B1 to B12, wherein the calcined catalyst particles, when contacted with propane under dehydrogenation conditions, generate a propylene yield of ≥48% at a propylene selectivity of ≥90%.

B14, The process of any one of B1 to B13, wherein a composition of the first atmosphere and a composition of second atmosphere independently remains constant or varies during the first time period and the second time period, respectively.

C1. A process for upgrading a hydrocarbon, comprising: subjecting a synthesized catalyst comprising Pt disposed on a support to an initial calcination comprising exposing the synthesized catalyst to a first reducing gas under reduction conditions or a first oxidizing gas under oxidation conditions to produce an initial calcined catalyst, wherein the synthesized catalyst comprises <0.05 wt % of the Pt, based on the non-volatile weight of the catalyst; optionally, subjecting the initial calcined catalyst to a cycle calcination comprising exposing the initial calcined catalyst to a second reducing gas under reduction conditions and a second oxidizing gas under oxidation conditions for n cycles to produce a cycle calcined catalyst, wherein: n is a whole number, the cycle calcination starts with the second oxidizing gas when the initial calcination uses the first reducing gas, the cycle calcination starts with the second reducing gas when the initial calcination uses the first oxidizing gas ; when n is ≤2, a composition of the second reducing gas used in each cycle calcination. is the same or different and a composition of the second oxidizing gas used in each cycle calcination is the same or different: and optionally, subjecting the initial calcined catalyst or the cycle calcined catalyst to a final calcination comprising exposing the initial calcined catalyst or the cycle calcined catalyst to a third reducing gas under reduction conditions or a third oxidizing gas under oxidation conditions, wherein: at least one of the cycle calcination and the final calcination is carried out, the final calcination, when carried out, uses the third oxidizing gas when the initial calcination uses the first reducing gas or, when carried out, the cycle calcination ends with the second reducing gas, the final calcination, when carried out, uses the third reducing gas when the initial calcination uses the first oxidizing gas or, when carried out, the cycle calcination ends with the second oxidizing gas, the reduction conditions used in the initial calcination, the optional cycle calcination, and the optional final calcination independently comprise heating the catalyst at a temperature in a range from 500° C. to 850° C. for a time period in a range from 30 seconds to 10 hours ; the oxidizing conditions used in the initial calcination, the optional cycle calcination, and the optional final calcination independently comprise heating the catalyst at a temperature in a range from 350° C. to 850° C. for a time period in a range from 30 seconds to 10 hours, and a calcined catalyst is obtained at the end of the cycle calcination or at the end of the final calcination; and contacting a hydrocarbon-containing feed with a calcined catalyst to effect one or more of dehydrogenation, dehydroaromatization, and dehydrocyclization of at least a portion of the hydrocarbon-containing feed to produce a coked catalyst composition and an effluent comprising one or more upgraded hydrocarbons and molecular hydrogen, wherein: the hydrocarbon-containing feed comprises one or more of C₂-C₁₆ linear or branched alkanes, or one or more of C₄-C₁₆ cyclic alkanes, or one or more C₈-C₁₆ alkyl aromatics, or a mixture thereof, the hydrocarbon-containing feed and calcined catalyst are contacted at a temperature in a range of from 300° C. to 900° C., for a time period of ≤3 hours, under a hydrocarbon partial pressure of at least 20 kPa-absolute, wherein the hydrocarbon partial pressure is the total partial pressure of any C₂-C₁₆ alkanes and any C₈-C₁₆ alkyl aromatics in the hydrocarbon-containing feed, and the one or more upgraded hydrocarbons comprise at least one of a dehydrogenated hydrocarbon, a dehydroaromatized hydrocarbon, and a dehydrocyclized hydrocarbon.

C2. The process of C1, further comprising: contacting at least a portion of the coked catalyst with an oxidant to effect combustion of at least a portion of the coke to produce a regenerated catalyst lean in coke and a combustion gas; and contacting an additional quantity of the hydrocarbon-containing feed with at least a portion of the regenerated catalyst to produce a re-coked catalyst and additional effluent, wherein a cycle time from contacting the hydrocarbon-containing feed with the calcined catalyst to contacting the additional quantity of the hydrocarbon-containing feed with the regenerated catalyst is ≤5 hours.

D1. A process for upgrading a hydrocarbon, comprising: subjecting synthesized catalyst particles comprising Pt disposed on a support to an initial calcination comprising exposing the catalyst particles to a first reducing gas under reduction conditions or a first oxidizing gas under oxidation conditions to produce initial calcined catalyst particles, wherein the synthesized catalyst particles have a size and particle density that is consistent with a Geldan A definition of a fluidizable solid; optionally, subjecting the initial calcined catalyst particles to a cycle calcination comprising exposing the initial calcined catalyst particles to a second reducing gas under reduction conditions and a second oxidizing gas under oxidation conditions for n cycles to produce cycle calcined catalyst particles, wherein: n is a whole number, the cycle calcination starts with the second oxidizing gas when the initial calcination uses the first reducing gas, the cycle calcination starts with the second reducing gas when the initial calcination uses the first oxidizing gas, when n is ≥2, a composition of the second reducing gas used in each cycle calcination is the same or different and a composition of the second oxidizing gas used in each cycle calcination is the same or different; and optionally, subjecting the initial calcined catalyst particles or the cycle calcined catalyst particles to a final calcination comprising exposing the initial calcined catalyst particles or the cycle calcined catalyst particles to a third reducing gas under reduction conditions or a third oxidizing gas under oxidation conditions, wherein: at least one of the cycle calcination and the final calcination is carried out, the final calcination, when carried out, uses the third oxidizing gas when the initial calcination uses the first reducing gas or, when carried out, the cycle calcination ends with the second reducing gas, the final calcination, when carried out, uses the third reducing gas when the initial calcination uses the first oxidizing gas or, when carried out, the cycle calcination ends with the second oxidizing gas, the reduction conditions used in the initial calcination, the optional cycle calcination, and the optional final calcination independently comprise heating the catalyst particles at a temperature in a range from 500° C. to 850° C. for a time period in a range from 30 seconds to 10 hours, the oxidizing conditions used in the initial calcination, the optional cycle calcination, and the optional final calcination independently comprise heating the catalyst particles at a temperature in a range from 350° C. to 850° C. for a time period in a range from 30 seconds to 10 hours, and calcined catalyst particles are obtained at the end of the cycle calcination or at the end of the final calcination; and contacting a hydrocarbon-containing feed with a calcined catalyst to effect one or more of dehydrogenation, dehydroaromatization, and dehydrocyclization of at least a portion of the hydrocarbon-containing feed to produce a coked catalyst composition and an effluent comprising one or more upgraded hydrocarbons and molecular hydrogen, wherein: the hydrocarbon-containing feed comprises one or more of C₂-C₁₆ linear or branched alkanes, or one or more of C₄-C₁₆ cyclic alkalies, or one or more C₈-C₁₆ alkyl aromatics, or a mixture thereof, the hydrocarbon-containing feed and calcined catalyst are contacted at a temperature in a range of from 300° C. to 900° C., for a time period of ≤3 hours, under a hydrocarbon partial pressure of at least 20 kPa-absolute, wherein the hydrocarbon partial pressure is the total partial pressure of any C₂-C₁₆ alkanes and any C₈-C₁₆ alkyl aromatics in the hydrocarbon-containing feed, and the one or more upgraded hydrocarbons comprise at least one of a dehydrogenated hydrocarbon, a dehydroaromatized hydrocarbon, and a dehydrocyclized hydrocarbon.

D2, The process of D1, further comptising: contacting at least a portion of the coked catalyst with an oxidant to effect combustion of at least a portion of the coke to produce a regenerated catalyst lean in coke and a combustion gas; and contacting an additional quantity of the hydrocarbon-containing feed with at least a portion of the regenerated catalyst to produce a re-coked catalyst and additional effluent, wherein a cycle time from contacting the hydrocarbon-containing feed with the calcined catalyst to contacting the additional quantity of the hydrocarbon-containing feed with the regenerated catalyst is ≤5 hours.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A process for calcining a catalyst, comprising:
subjecting a synthesized catalyst comprising Pt disposed on a support to an initial calcination comprising exposing the synthesized catalyst to a first reducing gas under reduction conditions or a first oxidizing gas under oxidation conditions to produce an initial calcined catalyst, wherein the synthesized catalyst comprises <0.05 wt % of the Pt, based on the non-volatile weight of the catalyst;
optionally, subjecting the initial calcined catalyst to a cycle calcination comprising exposing the initial calcined catalyst to a second reducing gas under reduction conditions and a second oxidizing gas under oxidation conditions for n cycles to produce a cycle calcined catalyst, wherein:
n is a whole number, the cycle calcination starts with the second oxidizing gas when the initial calcination uses the first reducing gas,
the cycle calcination starts with the second reducing gas when the initial calcination uses the first oxidizing gas,
when n is ≥2, a composition of the second reducing gas used in each cycle calcination is the same or different and a composition of the second oxidizing gas used in each cycle calcination is the same or different; and
optionally, subjecting the initial calcined catalyst or the cycle calcined catalyst to a final calcination comprising exposing the initial calcined catalyst or the cycle calcined catalyst to a third reducing gas under reduction conditions or a third oxidizing gas under oxidation conditions, wherein:
at least one of the cycle calcination and the final calcination is carried out,
the final calcination, when carried out, uses the third oxidizing gas when the initial calcination uses the first reducing gas or, when carried out, the cycle calcination ends with the second reducing gas,
the final calcination, when carried out, uses the third reducing vas when the initial calcination uses the first oxidizing gas or, when carried out, the cycle calcination ends with the second oxidizing gas,
the reduction conditions used in the initial calcination, the optional cycle calcination, and the optional final calcination independently comprise heating the catalyst at a temperature in a range from 500° C. to 850° C. for a time period in a range from 30 seconds to 10 hours,
the oxidizing conditions used in the initial calcination, the optional cycle calcination, and the optional final calcination independently comprise heating the catalyst at a temperature in a range from 350° C. to 850° C. for a time period in a range from 30 seconds to 10 hours, and a calcined catalyst is obtained at the end of the cycle calcination or at the end of the final calcination.

2. The process of claim 1, wherein the first oxidizing gas, if used, the second oxidizing gas and, if used, the third oxidizing gas independently comprise O₂, O₃, CO₂, steam, or a mixture thereof, and wherein the first reducing gas, if used, the second reducing gas, and, if used, the third reducing gas independently comprise H₂, CO, CH₄, C₄H₆, C₃H₈, C₂H₄, C₃H₆, steam, or a mixture thereof.

3. The process of claim 1, wherein:
the reduction conditions used in the initial calcination, the optional cycle calcination, and the optional final calcination independently comprise heating the catalyst at a temperature in a range from 550° C. to 700° C. for a time period in a range from 5 minutes to 1 hour, and
the oxidizing conditions used in the initial calcination, the optional cycle calcination, and the optional final calcination independently comprise heating the catalyst at a temperature in a range from 400° C. to 600° C. for a time period in a range from 5 minutes to 1 hour.

4. The process of claim 1, wherein the temperatures in the reduction conditions used in the initial calcination, the optional cycle calcination, and the optional final calcination are equal to or greater than the temperatures in the oxidizing conditions used in the initial calcination, the optional cycle calcination, and the optional final calcination.

5. The process of claim 1, wherein a sum of the time periods in the reduction conditions used in the initial calcination, the optional cycle calcination, and the optional final calcination is greater than a sum of the time periods in the oxidizing conditions used in the initial calcination, the optional cycle calcination, and the optional final calcination.

6. The process of claim 1, wherein, when the synthesized catalyst is subjected to the initial calcination, the synthesized catalyst comprises one or more volatile compounds, and wherein the one or more volatile compounds comprise adsorbed CO₂, adsorbed H₂O, adsorbed ethanol, or a mixture thereof.

7. The process of claim 1, wherein:
the synthesized catalyst further comprises up to 10 wt % of a promoter comprising Sn, Cu, Au, Ag, Ga, a combination thereof or a mixture thereof disposed on the support,
the synthesized catalyst comprises at least 0.5 wt % of a Group 2 element, and
all weight percent values are based on the non-volatile weight of the catalyst.

8. The process of claim 7, wherein:
the Group 2 element comprises Mg, and
at least a portion of the Group 2 element is in the form of MgO or a mixed metal oxide comprising Mg.

9. The process of claim 7, wherein:
the support further comprises a Group 13 element,
the promoter comprises Sn,
the Group 2 element comprises Mg,
the Group 13 element comprises Al, and
the support comprises a mixed Mg/A1 metal oxide.

10. The process of claim 1, wherein the synthesized catalyst is in the form of particles that have a size and particle density that is consistent with a Geldart A definition of a fluidizable solid.

11. The process of claim I, wherein the calcined catalyst, when contacted with propane under dehydrogenation conditions, generates a propylene yield of ≥48% at a propylene selectivity of ≥90%.

12. The process of claim 1, wherein the synthesized catalyst comprises 0.001 wt % to 0.045 wt % of the Pt, based on the non-volatile weight of the catalyst.

13. The process of claim 1, wherein the cycle calcination and the final calcination are both carried out.

14. The process of claim 1, wherein a composition of the first oxidizing gas, if used, a composition of the second oxidizing gas and, if used, a composition of the third oxidizing gas independently remains constant or varies during the initial calcination, during the cycle calcination, and during the final calcination, respectively.

15. The process of claim I, wherein a composition of the first reducing gas, if used, a composition of the second reducing gas and, if used, a composition of the third reducing gas independently remains constant or varies during the initial calcination, during the cycle calcination, and during the final calcination, respectively.

16. A process for calcining a catalyst, comprising:
subjecting synthesized catalyst particles comprising Pt disposed on a support to an initial calcination comprising exposing the catalyst particles to a first reducing gas under reduction conditions or a first oxidizing gas under oxidation conditions to produce initial calcined catalyst particles, wherein the synthesized catalyst particles have a size and particle density that is consistent with a Geldart A definition of a fluidizable solid;
optionally, subjecting the initial calcined catalyst particles to a cycle calcination comprising exposing the initial calcined catalyst particles to a second reducing gas under reduction conditions and a second oxidizing gas under oxidation conditions for n cycles to produce cycle calcined catalyst particles, wherein:
n is a whole number,
the cycle calcination starts with the second oxidizing gas when the initial calcination uses the first reducing gas,
the cycle calcination starts with the second reducing gas when the initial calcination uses the first oxidizing gas,
when n is ≥2, a composition of the second reducing gas used in each cycle calcination is the same or different and a composition of the second oxidizing gas used in each cycle calcination is the same or different; and
optionally, subjecting the initial calcined catalyst particles or the cycle calcined catalyst particles to a final calcination comprising exposing the initial calcined catalyst particles or the cycle calcined catalyst particles to a third reducing gas ander reduction conditions or a third oxidizing gas under oxidation conditions, wherein:
at least one of the cycle calcination and the final calcination is carried out,
the final calcination, when carried out, uses the third oxidizing gas when the initial calcination uses the first reducing gas or, when carried out, the cycle calcination ends with the second reducing gas,
the final calcination, when carried out, uses the third reducing gas when the initial calcination uses the first oxidizing gas or, when carried out, the cycle calcination ends with the second oxidizing gas,
the reduction conditions used in the initial calcination, the optional cycle calcination, and the optional final calcination independently comprise heating the catalyst particles at a temperature in a range from 500° C. to 850° C. for a time period in a range from 30 seconds to 10 hours,
the oxidizing conditions used in the initial calcination, the optional cycle calcination, and the optional final calcination independently comprise heating the catalyst particles at a temperature in a range from 350° C. to 850° C. for a time period in a range from 30 seconds to 10 hours, and
calcined catalyst particles are obtained at the end of the cycle calcination or at the end of the final calcination.

17. The process of claim 16, wherein the first oxidizing gas, if used, the second oxidizing gas and, if used, the third oxidizing gas independently comprise O₂, O₃, CO₂, steam, or a mixture thereof, and wherein the first reducing gas, if used, the second reducing gas, and, if used, the third reducing gas independently comprise H₂, CO, CH₄, C₂H₆, C₃H₈, C₂H₄, C₃H₆, steam, or a mixture thereof.

18. The process of claim 16, wherein:
the reduction conditions used in the initial calcination, the optional cycle calcination, and the optional final calcination independently comprise heating the catalyst particles at a temperature in a range from 550° C. to 700° C. for a time period in a range from 5 minutes to 1 hour, and
the oxidizing conditions used in the initial calcination, the optional cycle calcination, and the optional final calcination independently comprise heating the catalyst particles at a temperature in a range from 400° C. to 600° C. for a time period in a range from 5 minutes to 1 hour.

19. The process of claim 16, wherein the temperatures in the reduction conditions used in the initial calcination, the optional cycle calcination, and the optional final calcination are equal to or greater than the temperatures in the oxidizing conditions used in the initial calcination, the optional cycle calcination, and the optional final calcination.

20. The process of claim 16, wherein a sum of the time periods in the reduction conditions used in the initial calcination, the optional cycle calcination, and the optional final calcination is greater than a sum of the time periods in the oxidizing conditions used in the initial calcination, the optional cycle calcination, and the optional final calcination.

21. The process of claim 16, wherein, when the synthesized catalyst particles are subjected to the initial calcination, the synthesized catalyst particles comprise one or more volatile compounds, and wherein the one or more volatile compounds comprise adsorbed CO₂, adsorbed H₂O, adsorbed ethanol, or a mixture thereof.

22. The process of claim 16, wherein:
the synthesized catalyst particles further comprise up to 10 wt % of a promoter comprising Sn, Cu, Au, kg, Ga, a combination thereof, or a mixture thereof disposed on the support,
the synthesized catalyst particles comprises at least 0.5 wt % of a Group 2 element, and
all weight percent values are based on the non-volatile weight of the catalyst.

23. The process of claim 22, wherein:
the Group 2 element comprises Mg, and
at least a portion of the Group 2 element is in the form of MgO or a mixed metal oxide comprising Mg.

24. The process of claim 22, wherein:
the support further comprises a Group 13 element,
the promoter comprises Sn,
the Group 2 element comprises Mg,
the Group 13 element comprises A1, and
the support comprises a mixed Mg/Al metal oxide.

25. The process of claim 16, wherein the calcined catalyst particles, when contacted with propane under dehydrogenation conditions, generate a propylene yield of ≥48% at a propylene selectivity of ≥90%.

26. The process of claim 16, wherein the synthesized catalyst particles comprise 0.001 wt % to 6 wt % of the Pt, based on the non-volatile weight of the catalyst.

27. The process of claim 16, wherein the cycle calcination and the final calcination are both carried out.

28. The process of claim 16, wherein a composition of the first oxidizing gas, if used, a composition of the second oxidizing gas and, if used, a composition of the third oxidizing gas independently remains constant or varies during the initial calcination, during the cycle calcination, and during the final calcination, respectively.

29. The process of claim 16, wherein a composition of the first reducing gas, if used, a composition of the second reducing gas and, if used, a composition of the third reducing gas independently remains constant or varies during the initial calcination, during the cycle calcination, and during the final calcination, respectively.
